(19) United States
(12) Patent Application Publication
(10) Pub. No.: US 2026/0139275 A1
(43) Pub. Date: May 21, 2026

(54) STEM CELL BASED DELIVERY OF TUMOR-SPECIFIC RETROVIRAL VECTORS

(71) Applicants: The Regents of the University of California, Oakland, CA (US); Abintus Bio, Inc., San Diego, CA (US)

(72) Inventors: Noriyuki Kasahara, San Francisco, CA (US); Maria Barcova, San Francisco, CA (US); Sara A. Collins, San Francisco, CA (US); Akihito Inagaki, San Francisco, CA (US); Douglas Jolly, San Diego, CA (US)

(21) Appl. No.: 19/121,953

(22) PCT Filed: Oct. 17, 2023
(86) PCT No.: PCT/US2023/077104

§ 371 Date: Apr. 17, 2025

Related U.S. Application Data
(60) Provisional application No. 63/416,875, filed on Oct. 17, 2022.

(51) Int. Cl. C12N 15/867; A61K 35/28; A61K 45/06; C07K 14/16; C12N 5/0775; C12N 7/00; C12N 9/06; C12N 9/12; C12N 15/113

(52) U.S. Cl. CPC C12N 15/8673; A61K 35/28; A61K 45/06; C07K 14/161; C07K 14/162; C12N 5/0662; C12N 7/00; C12N 9/0044; C12N 9/1211; C12N 15/113; C12Y 107/01; C12Y 207/01021; C12N 2310/122; C12N 2510/04; C12N 2740/15043; C12N 2830/002; C12N 2830/34

(57) ABSTRACT

Provided herein are immortalized mesenchymal stem cells comprising a replicating recombinant retrovirus. Methods for treating a cell proliferative disorder using these immortalized mesenchymal stem cells are also provided.

### PRIOR RELATED APPLICATION

This application claims the benefit of and priority to U.S. Provisional Application No. 63/416,875 filed on Oct. 17, 2022, which is hereby incorporated by reference in its entirety.

### BACKGROUND

The American Cancer Society estimates 16,500 malignant brain tumors are diagnosed yearly in the United States, and account for 13,000 deaths (7,100 men & 5,900 women) per year. Malignant brain tumors represent 2.5% of adult cancer deaths and are the most common solid tumors in children. Furthermore, metastasis of systemic cancers to the brain is equally incurable, and this patient population is almost 10 times larger, with over 200,000 cases of brain-metastatic cancer each year in the U.S., accounting for 20% of all cancer deaths annually, and ˜10% of pediatric CNS tumors. In fact, as treatments improve for malignancies in other parts of the body, the brain becomes a ‘sanctuary site’ where cancer cells can escape and treatments cannot penetrate. With the increasing incidence of brain-metastatic lung, breast, skin, and blood cancers there is a need for new and effective therapies that target diffusely infiltrating cancer cells in the brain.

### SUMMARY

The inventors have discovered that immortalized mesenchymal cells (MSCs) (e.g., human mesenchymal stem cells) can be used as carriers for self-replicating retroviral viruses for gene therapy of proliferative disorders (e.g., brain cancer). This MSC-based carrier system significantly improves viral vector delivery to the CNS, as MSCs can actively migrate to diffusely infiltrating nests of tumor cells within the CNS and deliver the virus.

Provided herein is an immortalized mesenchymal stem cell comprising a replicating retrovirus (RRV), wherein the RRV comprises: (a) a nucleic acid encoding a retroviral gag protein, a retroviral pol protein and a retroviral env protein; and (b) an expression cassette comprising a first regulatory sequence operably linked to a first heterologous nucleic acid sequence encoding a heterologous polypeptide.

In some embodiments, the replicating retrovirus is selected from the group consisting of, murine leukemia virus (MLV), Moloney murine leukemia virus (MoMLV), Gibbon ape leukemia virus (GALV), Feline Leukemia virus (FeLV), RD114, and xenotropic XMLV. In some embodiments, the mesenchymal stem cell comprises a non-replicating lentivirus comprising an expression cassette comprising a second regulatory sequence operably linked to a second heterologous nucleic acid sequence encoding L-MYC and/or TERT. In some embodiments, the mesenchymal stem cell has reduced Class I HLA expression as compared to a naïve mesenchymal stem cell.

In some embodiments, the first heterologous nucleic acid sequence encodes a prodrug activator. In some embodiments, the prodrug activator is selected from the group consisting of a cytosine deaminase, a herpes simplex virus thymidine kinase, nitroreductase and cytochrome P450.

In some embodiments, the second heterologous nucleic acid further comprises a nucleic acid sequence encoding a selectable marker. In some embodiments, the first or second regulatory nucleic acid sequence is selected from the group consisting of a promoter, an enhancer, and an internal ribosome entry site. In some embodiments, the promoter is a constitutive or an inducible promoter.

Also provided is a method for making an immortalized mesenchymal stem cell comprising a replicating retrovirus (RRV), comprising: (a) transducing the immortalized mesenchymal stem cell with a replicating retrovirus (RRV), wherein the RRV comprises: (i) nucleic acid encoding a retroviral gag protein a retroviral pol protein and a retroviral env protein; and (ii) an expression cassette comprising a first regulatory sequence operably linked to a first heterologous nucleic acid sequence encoding a heterologous polypeptide; and (b) immortalizing the mesenchymal stem cell.

In some embodiments, the retrovirus is selected from the group consisting of, murine leukemia virus (MLV). Moloney murine leukemia virus (MoMLV). Gibbon ape leukemia virus (GALV), Feline Leukemia virus (FeLV). RD114, and xenotropic XMLV.

In some embodiments, immortalization comprises transducing the mesenchymal stem cell with a non-replication lentivirus comprising a second heterologous nucleic acid sequence encoding L-MYC and/or TERT. In some embodiments, the method further comprises transducing the mesenchymal stem cell with a nucleic acid sequence that reduces Class I HLA expression in the mesenchymal cell. In some embodiments, the nucleic acid sequence that reduces Class I HLA expression is a short hairpin RNA.

In some methods, the first heterologous nucleic acid sequence in the RRV encodes a prodrug activator. In some methods, the prodrug activator is selected from the group consisting of a cytosine deaminase, a herpes simplex virus thymidine kinase, nitroreductase and cytochrome P450. In some methods, the first or second regulatory nucleic acid sequence is selected from the group consisting of a promoter, an enhancer, and an internal ribosome entry site. In some methods, the promoter is a constitutive or an inducible promoter.

Also provided is an immortalized mesenchymal stem cell or population of immortalized mesenchymal stem cells produced by any of the methods described herein.

Further provided is a pharmaceutical composition comprising any of the immortalized mesenchymal stem cell or population of immortalized mesenchymal stem cells described herein.

Also provided is a method of treating a disease in a subject in need thereof comprising administering any of the immortalized mesenchymal stem cells or population of immortalized mesenchymal stem cells described herein, or any pharmaceutical composition described herein.

In some methods, the disease is a cell proliferative disorder. In some methods, the cell proliferative disorder is selected from the group consisting of lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, and brain cancer including glioblastoma, anaplastic astrocytoma, oligodendroglioma, medulloblastoma. In some methods, the cancer is glioblastoma.

In some methods, the subject is a mammal. In some methods, the subject is a human. In some methods, administration is systemic, topical or local administration. In some embodiments, the method further comprises administering a second therapeutic agent to the subject. In some embodiments, the second therapeutic agent is a nontoxic prodrug that is converted to a toxic drug by the prodrug activator. In some embodiments, the second therapeutic agent is a chemotherapeutic agent.

### BRIEF DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows that naïve MSCs migrated in vivo into the established intracranial human U87 glioma xenograft lesions from the contralateral injection side in athymic nude mice. 7 days after intracranial implantation of U87 glioma cells (2×10⁵ cells), CellTracker™ CM-DiI Dye-prelabeled MSC (5×10⁴ cells) were injected into the contralateral hemisphere. Brain tissues were harvested 10 days later and MSC migratory activity was examined by histology of frozen 50-μm sections.

FIG. 2 shows tumor transduction by naïve human MSC-RRV (GFP) producer cells vs. RRV virus in U87 s.c. tumor model in athymic nude mice. RRV (GFP) (2×10³ infectious units) or CM-DiL-labeled MSC-RRV (GFP)-cells (2×10³ RRV producer cells), were injected s.c. when tumor reached 8-mm diameter. Tumor was excised 5 days later and brain sections were examined by fluorescence microscopy. The area of GFP positive cells showed multiple foci and higher transduction efficiency after MSC-RRV injection. In contrast. RRV bolus injection resulted in a single area of GFP positive cells, occupying half of the tumor cross section. qPCR analysis of vector copy number/cell shows higher transduction by MSC-RRV compared to single bolus injection of RRV alone.

FIG. 3 shows that naïve human MSC-RRV (CD) producer cells significantly prolonged survival in an intracranial U87 human glioma model. Results were obtained after 1 cycle of 5-FC prodrug, in a U87 human glioma xenograft model (established with 2×10⁵ glioma cells for 14 days) injected with human MSC-RRV (CD) (2×10³ RRV producer cells) vs. RRV-CD (Toca511) virus (2×10³ infectious units).

FIG. 4 is a schematic of a RRV comprising a nucleic acid sequence encoding gag, pol, env and green fluorescent protein (GFP) or cytosine deaminase (CD).

FIG. 5 provides schematics for several non-replicating lentiviral (LV) constructs: LV-ELIN, LV-ETIP, LV-shABC-ER, and LV-shABC-ELTIN, as described in the Examples.

FIG. 6 is a graph showing expression of L-MYC in transduced Wharton's Jelly (WJ)-MSC cells relative to untransduced naïve cells. qPCR analysis of L-myc expression was normalized to RNaseP.

FIG. 7 is a graph showing expression of hTERT in transduced WJ-MSC cells relative to untransduced naïve cells. qPCR analysis of hTERT expression was normalized to RNaseP.

FIG. 8A is a graph showing growth rate, as population doubling time (PDT), of L-MYC/hTERT transduced WJ-MSC cells. Cells were passaged at 5000/cm² for 96 hours. Clonal immortalized cell line ALT422 stably maintained its robust self-renewal capabilities, can be propagated up to at least passage 27, and expands robustly (15.4-fold in 4 days in passage 16). In comparison, naïve WJ-MSC cells expanded only 1.6-fold in passage 16 when they reached senescence.

FIG. 8B is a graph showing that clonal cell line ALT422 stably maintains its robust self-renewal capabilities at PDT averaging 30.4 hours, and can be propagated up to at least passage 52. Compared to clone ALT422, clone LT100 maintained its PDT at the average PDT 32.5 hours starting from passage 6, when it emerged, to at least passage 45.

FIG. 9 is a graph showing daily growth rate as cell number of L-MYC/hTERT transduced clone ALT422 vs. naïve WJ-MSC cells at passage 15. Cells were plated at 5×10⁴ cells/well in 6-well plate, on day 0.

FIG. 10 is a graph showing the activity of the senescence-associated β-galactosidase in L-MYC/hTERT transduced MSC-RRV clones.

FIG. 11 shows representative images of SA-β-GAL stained MSC cells at 200× magnification.

FIG. 12 is a graph showing expression of senescence marker CDKN2A/p16 in transduced WJ-MSC cells relative to untransduced naïve cells. qPCR analysis of p16 expression was normalized to RNaseP.

FIG. 13 shows the results of an in vitro tumorigenicity test comparing the ALT422 clone with the U87 glioma cells line. The images represent 14 days of growth in 1% agarose hydrogel.

FIG. 14 is a graph showing 48-hr migration of L-MYC/hTERT clones across matrigel-coated 8-mm-pore size transwells towards serum-free supernatant of patient-derived glioma NCT cells. 1×10⁵ cells/transwell, in a 24 well plate, were used.

FIG. 15 shows representative images of propidium-iodide-stained MSC cells that migrated across the Matrigel-coated transmembrane in Boyden chamber towards the serum-free (SF) medium or SF-conditioned medium of patient-derived glioma cell line NCT (NCT-CM) at 200× magnification.

FIG. 16 shows the ALT422 cells labeled with LV (mCherry) for use in the coculture experiments with NCT cells. Image shows the overlap of green and red fluorescence. ALT422 cells produce high RRV (GFP) vector titer 5.9×10⁶ TU/ml.

FIG. 17a-e show a four-day RRV-GFP virus spread in a mixed cell coculture at 1:100 ratio of immortalized ALT422 MSC-RRV producer cells to patient-derived NCT glioma cells.

FIG. 18 shows that ALT422 cells are positive for all MSC markers and negative for hematopoietic lineage markers.

FIG. 19 shows MSC-RRV-shABC clones 1, 2 and 3 generated by RRV-yCD (vector Toca 511) infection of WJ-MSCs followed by LV-shABC-ELTIN (schematics in FIG. 5) transduction. Individual G418-resistant clones show 35, 44 and 46% knockdown of Class-I HLA antigens, compared to RRV-yCD-infected MSCs not transduced with LV-shABC-ELTIN vector.

FIG. 20 is a G-banded metaphase spread of the human cell line ALT422 showing that ALT422 has a normal female karyotype 46, XX.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### INTRODUCTION

Despite modern advances in treatment, patients afflicted with cancers such as glioblastoma, the most malignant type of primary brain tumor, continue to face a dire prognosis. Although viral therapies such as retrovirus-based replicating vectors (RRVs) have been developed, these vectors have limitations. To administer RRV into brain tumor patients, the virus has generally been injected into the center of the tumor, or into the walls of the tumor rescetion cavity. Yet, human brain tumors often diffusely infiltrate into surrounding normal brain, making them difficult to eradicate by locally administered agents, even when using a self-replicating virus. Since the virus is not motile, initial infection is dependent on simple diffusion, before the virus progressively infects by cell-to-cell spread. Hence, tissue penetration and infection are limited at first, which delays spread of the virus to outlying tumor nests. Provided herein are compositions and methods for stem-cell based delivery of RRVs that improve initial infection efficiency of multi-focal brain tumors and accelerate the kinetics of intra-tumoral RRV dissemination in vivo, as compared to injecting the RRV by itself.

### Definitions

As used in this specification and the appended claims, the singular forms “a,” “an,” and “the” include plural reference unless the context clearly dictates otherwise.

“About” is used to provide flexibility to a numerical range endpoint by providing that a given value may be “slightly above” or “slightly below” the endpoint without affecting the desired result.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The use herein of the terms “including.” “comprising,” or “having,” and variations thereof, is meant to encompass the elements listed thereafter and equivalents thereof as well as additional elements. Embodiments recited as “including.” “comprising.” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. Retroviruses are enveloped single-stranded RNA viruses that typically infect mammals, such as, for example, bovines, monkeys, sheep, and humans, as well as avian species. Retroviruses are unique among RNA viruses in that their multiplication involves the synthesis of a DNA copy of the RNA which is then integrated into the genome of the infected cell. As used herein, “a self-replicating retrovirus or replication retroviral vector (RRV)” is a retrovirus encoding all viral proteins necessary for viral replication (i.e., a retroviral gag protein, a retroviral pol protein and a retroviral env protein). The nucleic acid sequence encoding the gag. pol and env proteins are flanked by a 5′ LTR and a 3″ LTR. The gag gene encodes the internal structural (matrix, capsid, and nucleocapsid) proteins: the pol gene encodes the RNA-directed DNA polymerase (reverse transcriptase), protease and integrase; and the env gene encodes viral envelope glycoproteins. The 5′ and 3′ LTRs of the retrovirus serve to promote transcription and polyadenylation of the virion RNAs. Lentiviruses have additional genes including vif, vpr, tat, rev, vpu, nef, and vpx (in HIV-1, HIV-2 and/or SIV). See, also, U.S. Pat. Nos. 6,410,313, 6,899,871, and 8,741,279, incorporated herein in their entireties by this reference, for additional information regarding self-replicating retroviruses.

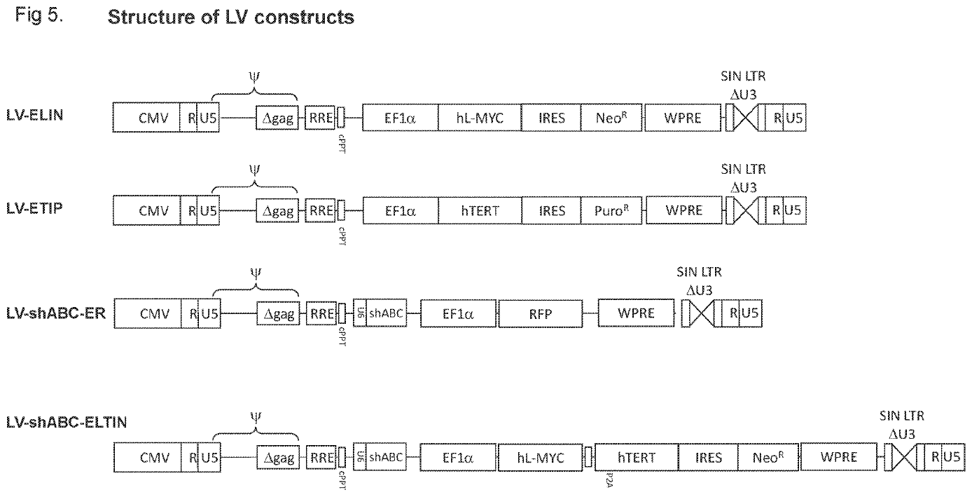

The Retroviridae family consists of three groups: the spumaviruses (or foamy viruses) such as the human foamy virus (HFV); the lentiviruses, as well as visna virus of sheep; and the oncoviruses (although not all viruses within this group are oncogenic). The term “retrovirus” is used in its conventional sense to describe a genus of viruses containing reverse transcriptase. Retroviruses include lentiviruses. As used herein, a lentiviral vector is the lentivirus which include the “immunodeficiency viruses” which include human immunodeficiency virus (HIV) type 1 and type 2 (HIV-1 and HIV-2) and simian immunodeficiency virus (SIV). The oncoviruses are further subdivided into groups A, B, C and D on the basis of particle morphology, as seen under the electron microscope during viral maturation. Lentiviruses include non-replicating or replication deficient lentiviruses. In some embodiments, the lentivirus is rendered replication incompetent by modifying the lentivirus to comprise a modified (e.g., a U3 deletion (ΔU3)), self-inactivating (SIN) 3′ LTR which renders the resulting lentiviral particles replication incompetent. See, FIG. 5 for exemplary replication incompetent lentiviral constructs.

Retroviruses are defined by the way in which they replicate their genetic material. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virion.

The retrovirus particle is composed of two identical RNA molecules. Each wild-type genome has a positive sense, single-stranded RNA molecule, which is capped at the 5′ end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer, derived from host cell membranes and containing viral envelope (env) proteins. The env proteins bind to a cellular receptor for the virus and the particle typically enters the host cell via receptor-mediated endocytosis and/or membrane fusion. After the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into the more complex DNA genome. The double-stranded linear DNA produced by reverse transcription may, or may not, have to be circularized in the nucleus. The provirus now has two identical repeats at either end, known as the long terminal repeats (LTR). The termini of the two LTR sequences produces the site recognized by a pol product, the integrase protein, which catalyzes integration, such that the provirus is always joined to host DNA two base pairs (bp) from the ends of the LTRs. A duplication of cellular sequences is seen at the ends of both LTRs. Integration is thought to occur essentially at random within the target cell genome. However, by modifying the long terminal repeats it is possible to control the integration of a retroviral genome.

Transcription, RNA splicing, and translation of the integrated viral DNA is mediated by host cell proteins. Efficient infectious transmission of retroviruses requires the expression on the target cell of receptors which specifically recognize the viral envelope proteins, although viruses may use receptor-independent, nonspecific routes of entry at low efficiency. In addition, the target cell type must be able to support all stages of the replication cycle after virus has bound and penetrated.

As used herein, “a viral vector” refers to a gene therapy vector used to deliver a polynucleotide construct to a cell. It is understood that the term viral vector encompasses recombinant vector particles or virions (i.e., viral particles comprising at least one capsid or envelope protein and an encapsidated recombinant viral vector) and recombinant vector plasmids.

As used herein, a “recombinant viral vector” refers to a viral vector, for example, retroviral vector comprising a nucleic acid sequence that is not normally present in the viral vector (i.e., a polynucleotide heterologous to the viral vector). In general, the heterologous nucleic acid, for example, a nucleic acid encoding a heterologous polypeptide, is flanked by at least one, and generally by two, long terminal repeat sequences (LTRs), for example, a 5′ LTR and a 3′LTR. As used herein, the retroviral vector can be a derivative of a murine, simian or human retrovirus. Examples of retroviral vectors in which a transgene (e.g., a heterologous polynucleotide sequence) can be inserted include, but are not limited to lentivirus. Moloney murine leukemia virus (MoMuLV). Harvey murine sarcoma virus (HaMuSV), murine mammary tumor virus (MuMTV), Rous Sarcoma Virus (RSV), murine leukemia virus (MLV), Gibbon ape leukemia virus (GALV), Feline Leukemia virus (FeLV). RD114, and xenotropic XMLV.

The term “nucleic acid” or “nucleotide” refers to deoxyribonucleic acids (DNA) or ribonucleic acids (RNA) and polymers thereof, for example, polynucleotides, in either single-or double-stranded form. The nucleic acid molecule may be derived from a variety of sources, including DNA, cDNA, synthetic DNA, RNA, or combinations thereof. Such nucleic acid sequences may comprise genomic DNA which may or may not include naturally occurring introns. Moreover, such genomic DNA may be obtained in association with promoter regions, introns, or poly A sequences.

Unless otherwise indicated, a particular nucleic acid sequence also implicitly encompasses conservatively modified variants thereof (e.g., degenerate codon substitutions), alleles, orthologs. SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” or “transgene” can refer to the segment of DNA (e.g., a polynucleotide sequence) involved in producing or encoding a polypeptide chain. It may include regions preceding and following the coding region (leader and trailer) as well as intervening sequences (introns) between individual coding segments (exons). Alternatively, the term “gene” or “transgene” can refer to the segment of DNA involved in producing or encoding a non-translated RNA, such as an rRNA, tRNA, guide RNA (e.g., a single guide RNA), or micro RNA.

As used herein the phrase “heterologous” refers to what is not normally found in nature. The term “heterologous nucleotide sequence” refers to a nucleotide sequence not normally found in a given wild-type viral genome, or a cell in nature. As such, a heterologous nucleotide sequence may be: (a) foreign to its host cell (i.e., is exogenous to the cell): (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide.” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the phrase “introducing” in the context of introducing a nucleic acid or a viral vector refers to the translocation of the nucleic acid sequence or viral vector from outside a cell to inside the cell. In some cases, introducing refers to transducing or infecting a cell or a population of cells with a viral vector or viral particle carrying one or more non-viral nucleic acids. In some cases, translocation of the nucleic acid from outside the cell to inside the nucleus of the cell occurs. Various methods of such translocation are contemplated, including but not limited to, viral infection, electroporation, transfection, transduction, contact with nanowires or nanotubes, receptor mediated internalization, translocation via cell penetrating peptides, liposome mediated translocation, and the like.

As used herein, a “cell” can be in vivo, ex vivo or in vitro, and includes any cell that can be transduced or infected by a retrovirus, for example, a human MSC. As used herein the term “mesenchymal stem cell” or “MSC” refers to multipotent stem cells found in bone marrow that are important for making and repairing skeletal tissues, such as cartilage, bone and the fat found in bone marrow. In some embodiments, the cells are human mesenchymal stem cells.

In some cases, the cell infected by a retrovirus is an immortalized human MSC. In some cases, primary MSC cells are isolated from an organism, system, organ, or tissue, optionally sorted, and immortalized. In some embodiments, the primary MSC cells are immortalized by transducing the mesenchymal stem cell with a nucleic acid sequence encoding and oncogene, for example, L-MYC and/or telomerase reverse transcriptase (TERT). In some embodiments, the primary MSC cells are transduced with a non-replication lentivirus comprising a second heterologous nucleic acid sequence encoding L-MYC and/or TERT. In some embodiments, the MSC is transduced with a RRV and a non-replicating lentivirus described herein prior to immortalization.

In some cases, the primary cells are stimulated, activated, or differentiated prior to immortalization. As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In contrast, immortalized cells can proliferate indefinitely, and can be cultured, sub-cultured, or passaged at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times or more. As used herein, the term “naïve” refers to a cell, for example, an MSC, that has not been immortalized.

### Compositions

Clinical use of autologous patient derived MSCs is hindered by low yield of harvested MSCs, requiring ex vivo expansion that is limited to 30-40 population doublings due to the replicative senescence phenomenon in primary MSCs. To circumvent these inherent constraints on scalability of primary human MSCs, the inventors generated an immortalized “universal-donor” MSC-RRV clonal cell line, for delivery of RRV, that is positive for human MSC markers, is not tumorigenic, and retains self-renewal capabilities.

Provided herein is an immortalized mesenchymal stem cell comprising a replicating retrovirus (RRV), i.e., an MSC-RRV, wherein the RRV comprises: (a) a nucleic acid encoding a retroviral gag protein, a retroviral pol protein and a retroviral env protein; and (b) an expression cassette comprising a first regulatory sequence operably linked to a first heterologous nucleic acid sequence encoding a heterologous poly peptide Populations of any of the immortalized cells described herein are also provided.

In some embodiments, the replicating retrovirus is selected from the group consisting of, murine leukemia virus (MLV), Moloney murine leukemia virus (MoMLV), Gibbon ape leukemia virus (GALV).

In some embodiments, the mesenchymal stem cell comprises a non-replicating lentivirus comprising an expression cassette comprising a second regulatory sequence operably linked to a second heterologous nucleic acid sequence encoding L-MYC and/or TERT. In some embodiments, the mesenchymal stem cell has reduced Class I HLA expression as compared to a naïve (i.e., non-immortalized) mesenchymal stem cell. Exemplary lentiviruses comprising an expression cassette comprising a second regulatory sequence operably linked to a second heterologous nucleic acid sequence encoding L-MYC and/or TERT are shown in FIG. 5. In the immortalized MSC-RRV cells described herein the first heterologous nucleic acid sequence and the second heterologous nucleic acid sequence, i.e., the nucleic acid encoding one or more oncogenes, for example, L-MYC and/or TERT, are encoded by the replicating retrovirus and the non-replicating lentivirus, respectively. It is understood that one or more oncogenes used for immortalization, for example, L-MYC and/or TERT, cannot be encoded by the replicating virus that encodes the first heterologous polypeptide, for example, a pro-drug activator. Expressing the nucleic acid sequence encoding the oncogene(s) from a separate, non-replicating virus prevents mobilization of the oncogene-encoding provirus by the replicating virus.

In the MSC-RRV cells described herein, the first heterologous nucleic acid sequence can encode any polypeptide of interest, such as transcription factors and cytokines that are negative regulators of tumor microenvironment, prodrug activators, or truncated versions of surface molecules, such as truncated human epithelial growth factor receptor (tEGFR) polypeptide for targeting by cetuximab) (Wang et al. Gene Therapy 118 (5): 1255-1263 (2011)). See, for example, Wang et al. Blood 118 (5): 1255-1263 (2011)). In some embodiments, the first heterologous nucleic acid sequence encodes a prodrug activator. See, for example. Sheikh et al. “Prodrugs and prodrug-activated systems in gene therapy.” Molecular Therapy 29 (5): 1716-1728 (2021) In cases where a prodrug activator is used, a nontoxic prodrug can be administered to the subject, such that when the activator is expressed, conversion of the nontoxic prodrug into a toxic drug, (i.e, a cell-killing drug), takes place. Common enzymes that are used in prodrug-activated systems are herpes simplex virus (HSV)-derived thymidine kinase (TK: HSV-TK), cytosine deaminase (CD) from Escherichia coli or yeast, and E. coli-associated nitroreductase (NTR), which render cells sensitive to their respective prodrugs, ganciclovir (GCV), 5-fluorocytosine (5-FC), and CB1954.

In addition to being tumor-specific, MSC-RRV has a built-in, internal safeguard mechanism against the potential risk of uncontrolled in vivo spread and/or malignant transformation in a subject. The mechanism for this in vivo safeguard system is the RRV-encoded expression of a prodrug activator, for example, cytosine deaminase (CD) prodrug activator enzyme. MSC-RRV cells can be eliminated in vivo by administration of prodrug 5-fluorocytosine (5-FC) which converts the nontoxic prodrug into a toxic drug that kills the MSC-RRV cells. Therefore. MSC-RRV cells represent a therapeutic platform without systemic toxicity, since viral replication is tumor-selective and systemic administration of 5-FC is non-toxic.

In some embodiments, the second heterologous nucleic acid encoded by the non-replicating lentivirus further comprises a nucleic acid sequence encoding a selectable marker. As used herein, the term “selectable marker” refers to a gene which allows selection of a host cell comprising a marker. The selectable markers may include, but are not limited to: fluorescent markers, luminescent markers and drug selectable markers, cell surface receptors, and the like. In some embodiments, the selection can be positive selection; that is, the cells expressing the marker are isolated from a population, e.g., to create an enriched population of cells expressing the selectable marker. Separation can be by any convenient separation technique appropriate for the selectable marker used. For example, if a fluorescent marker is used, cells can be separated by fluorescence activated cell sorting, whereas if a cell surface marker has been inserted, cells can be separated from the heterogencous population by affinity separation techniques, e.g., magnetic separation, affinity chromatography, “panning” with an affinity reagent attached to a solid matrix, fluorescence activated cell sorting or other convenient technique. Other selection genes encode proteins that confer resistance to antibiotics and other toxic substances, e.g., histidinol, puromycin, hygromycin, neomycin, methotrexate, and other reporter genes known in the art.

As used herein, the term “regulatory nucleic acid sequence” refers collectively to promoter sequences, promoter/enhancer sequences, polyadenylation signals, transcription termination sequences, upstream regulatory domains, origins of replication, internal ribosome entry sites (“IRES”), enhancers, and the like, which collectively provide for the replication, transcription, and translation of a coding sequence in a recipient cell. Some or all of these regulatory sequences can be present as long as the selected coding sequence is capable of being replicated, transcribed and translated in an appropriate host cell. One skilled in the art can readily identify regulatory nucleic acid sequences from public databases and materials. Furthermore, one skilled in the art can identify a regulatory sequence that is applicable for the intended use, for example, in vivo, ex vivo, or in vitro use.

In some embodiments, the first or second regulatory nucleic acid sequence is selected from the group consisting of a promoter, an enhancer, and an internal ribosome entry site. In any of the compositions provided herein, the promoter in any of the vectors provided herein can be a constitutive promoter (e.g., SV40, EFIA, RSV, CMV, etc.) or an inducible promoter (e.g., tetracycline (Iida et al. J. Virol., 70 (9): 6054-9), GAL4 target upstream activating sequence (Osterwalder et al., PNAS 98 (22): 12596-12601 (2001). Cumate inducible expression system (Seo and Dannert, Appl. Microbiol. Biotechnol. 103 (1): 303-313 (2019)).

The promoter can also be a cell-specific or tissue-specific promoter. When using a cell- or tissue-specific promoter, viral replication occurs primarily, but not exclusively, in a particular cell or tissue. For example, viral replication can occur in at least 90%, 95%, or 99% of the targeted cell or tissue. It will be understood, however, that tissue-specific promoters may have a detectable amount of background or base activity in those tissues where they are mostly silent. The degree to which a promoter is selectively activated in a target tissue can be expressed as a selectivity ratio (activity in a target tissue/activity in a control tissue). In this regard, a tissue-specific promoter useful in the practice of the present invention typically has a selectivity ratio of greater than about 5. Preferably, the selectivity ratio is greater than about 15.

In some embodiments, the retroviral genome of the RRV and/or the non-replicating lentivirus contains an IRES comprising a cloning site for insertion of a desired polynucleotide sequence, preferably the IRES is 3′ to the env gene in the retroviral vector Accordingly, a heterologous poly nucleotide sequence encoding a desired poly peptide may be operably linked to the IRES An example of polynucleotide sequence which may be operably linked to the IRES include green fluorescent protein (GFP) or a selectable marker gene. Marker genes are utilized to assay for the presence of the vector, and thus, to confirm infection and integration Other polynucleotide sequences that can be linked to the IRES include, suicide genes, such as, for example, HSV-thymidine kinase, or polynucleotide sequences that encode an antisense molecule.

In some embodiments, the components of the RRV or the non-replication lentivirus, are expressed in multicistronic fashion, by including one or more self-cleaving peptides in between two or more nucleic acids to be expressed as a multicistronic, for example, a bicistronic sequence. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example. Chng et al. “Cleavage efficient 2A peptides for high level monoclonal antibody expression in CHO cells.” MAbs 7 (2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different.

In some embodiments, the immortalized MSC-RRV cells express a targeting moiety (e.g., an antibody, a ligand, peptide, etc.) on the cell surface to increase MSC homing to tumors. In some embodiments, the MSC-RRV cells express a ligand that is specific for a tumor receptor, for example a tumor cell marker erbB2. See, for example, Komaraova et. al., J. Ovarian Res. 3 (12): 1757-2215 (2010)).

In another embodiment, MSC-RRV cells express proteins that enhance migratory properties and mediate more targeted migration to the tumor sites. In some embodiments, MSC-RRV can be engineered to express podoplanin Although podoplanin does not act as a specific tumor receptor, this agent improves migration to tumor sites. See, for example, Danielyan et al. EBioMedicine. Volume 60, 2020, 102989.

In another embodiments, immortalized MSC-RRV cells can be modified to increase tumor tropism by, for example, incubating the MSCs with cytokines, such as, for example, TNF-α. For example, priming of bone marrow-derived MSC and adipose MSC with TNF-α in concentrations between 1 ng/ml to 100 ng/ml for 24 hrs was shown to increase MSC migration. SEe, for example, Stem Cells 25 (7): 1737-45 (2007). In some cases, the speed of in vivo MSC-RRV migration can also be enhanced by, for example, inhibiting LINC01773, a non-coding RNA in the MSC cells. See, for example. Chinese Patent No. CN112391386B. In some cases, the inhibitor is siRNA, shRNA, dsRNA or miRNA for reducing the expression level of LINC 01773.

The immortalized MSC-RRV cells described herein or a population of immortalized MSC-RRV cells described herein can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. The compositions will include a therapeutically effective amount of the immortalized MSC-RRV cells described herein in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained. Such pharmaceutically acceptable carriers include sterile biocompatible pharmaceutical carriers, including, but not limited to, saline, buffered saline, artificial cerebral spinal fluid, dextrose, and water.

### Methods of Making Immortalized MSC-RRV Cells

Provided herein is a method for making an immortalized mesenchymal stem cell comprising a replicating retrovirus (RRV), comprising: (a) transducing the mesenchymal stem cell with a replicating retrovirus (RRV), wherein the RRV comprises: (i) nucleic acid encoding a retroviral gag protein a retroviral pol protein and a retroviral env protein; and (ii) an expression cassette comprising a first regulatory sequence operably linked to a first heterologous nucleic acid sequence encoding a heterologous polypeptide; and (b) immortalizing the mesenchymal stem cell.

Also provided herein is a method for making an immortalized mesenchymal stem cell comprising a replicating retrovirus (RRV), comprising: (a) immortalizing the mesenchymal stem cell; and (b) transducing the immortalized mesenchymal stem cell with a replicating retrovirus (RRV), wherein the RRV comprises: (i) nucleic acid encoding a retroviral gag protein a retroviral pol protein and a retroviral env protein; and (ii) an expression cassette comprising a first regulatory sequence operably linked to a first heterologous nucleic acid sequence encoding a heterologous polypeptide. Any of the methods of making a population of immortalized MSC RRV cells provided herein can further comprise expanding the population of immortalized MSC-RRV cells.

In some embodiments, the retrovirus is selected from the group consisting of, murine leukemia virus (MLV), Moloney murine leukemia virus (MoMLV), Gibbon ape leukemia virus (GALV).

In some embodiments, immortalization comprises transducing the mesenchymal stem cell with a non-replication lentivirus comprising a second heterologous nucleic acid sequence encoding L-MYC (Addgene Plasmi ID 26022) and/or hTERT (Addgene plasmid ID 1774). Exemplary nucleic acids encoding L-MYC and hTERT are provided herein as SEQ ID NO: 1 and SEQ ID NO: 2, respectively. Nucleic acid sequences comprising SEQ ID NO: 1, 2, or 3 can be used in any of the non-replicating lentiviruses described herein. Nucleic acid sequences having at least about 80%, 85%, 90%, 95%, 99% identity to SEQ ID NO: 1, 2, or 3 can also be used.

In some embodiments, the method further comprises transducing the stem cell with a nucleic acid sequence that reduces Class I HLA expression in the mesenchymal cell. In some embodiments, the nucleic acid sequence that reduces Class I HLA expression is siRNA, shRNA, dsRNA or miRNA. See, for example, Haga et al. Tranpslant Proc. 38 (10): 3184-8 (2006)). In some embodiments, the nucleic acid sequence that reduces Class I HLA expression is expressed from the non-replicating lentiviral vector. In some embodiments, the shRNA that reduces Class I HLA expression comprises SEQ ID NO: 3 or a sequence having at least 60%, 65%, 70%, 75%, 80%, 85%, 90%, 95%, or 99% identity to SEQ ID NO: 3. SEQ ID NO: 3 is an exemplary shRNA as other shRNAs that reduce Class I HLA expression can be generated/designed by one of skill in the art using routine methods known in the art.

Reduction of self-antigen expression makes the cells less immunogenic, thus enabling production of universally compatible, “off-the-shelf” MSC-RRV, for adoptive cell transfer to allogeneic cancer patients without immunological rejection. As used herein, the reduction in self antigen expression can be at least 10%, 20%, 30%, 40%, 50%, 60%, 70%. 80%, 90% or 100%.

In some methods, the first heterologous nucleic acid sequence in the RRV encodes a prodrug activator. In some methods, the prodrug activator is selected from the group consisting of a cytosine deaminase, a herpes simplex virus thymidine kinase, nitroreductase and cytochrome P450. In some methods, the first or second regulatory nucleic acid sequence is selected from the group consisting of a promoter, an enhancer, and an internal ribosome entry site. In some methods, the promoter is a constitutive or an inducible promoter as described above

### Methods

Also provided are methods for using any of the immortalized MSC-RRV cells or populations of immortalized MSC-RRV cells provided herein to efficiently deliver transgenes to tumors in vivo for therapeutic purposes.

Provided herein is a method of treating a disease in a subject in need thereof comprising administering a therapeutically effective amount of any of the immortalized mesenchymal stem cells or population of immortalized mesenchymal stem cells described herein, or a therapeutically effective amount of any pharmaceutical composition described herein.

In any of the methods provided herein, the subject can be a subject diagnosed with a disease, for example, a cell proliferative disorder. “Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; preventing a relapse, or making the final point of degeneration less debilitating. For example, a method for treating cancer is considered to be a treatment if there is a 10% reduction in one or more symptoms of the cancer in a subject as compared to a control Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

Any of the methods provided herein can be used to treat a cell proliferative disorder. The term “cell proliferative disorder” refers to a condition characterized by an abnormal number of cells. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but can include normal cells as well.

In some methods, the cell proliferative disorder is selected from the group consisting of lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, and brain cancer including glioblastoma, anaplastic astrocytoma, oligodendroglioma, medulloblastoma. In some methods, the cancer is glioblastoma.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder. The term patient or subject includes human and veterinary subjects.

Any of the methods provided herein can further comprise administering a second therapeutic agent to the subject. The second therapeutic agent can be selected from the group consisting of a chemotherapeutic agent, an adjuvant, an immunomodulatory agent, a vaccine, a tumor antigen, or a combination thereof. In some instances, the second therapeutic agent is a prodrug that can be converted into a toxic drug by a prodrug activator encoded by the RRV. Administration of a non-toxic prodrug is useful in situations where it is desirable to eliminate the MSC-RRV cells after administration of the cells to the subject.

In cases, where the second therapeutic is a nucleic acid sequence encoding a therapeutic polypeptide, the second therapeutic agent can be delivered by viral or non-viral means.

Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ (2) and combinations thereof.

It is understood that combinations, for example, a composition comprising immortalized MSC RRV cells described herein, and a second therapeutic agent can be administered either concomitantly (e.g., as an admixture), separately but simultaneously (e.g., via separate intravenous lines into the same subject), or sequentially (e.g., one of the compositions or agents is given first followed by the second). Any of the methods provided herein can further comprise radiation therapy or surgery.

As used herein, the term “therapeutically effective amount” or “effective amount” refers to an amount of a composition that, when administered to a subject, is effective, alone or in combination with additional agents, to treat a disease or disorder either by one dose or over the course of multiple doses. A suitable dose can depend on a variety of factors including the particular composition or system used and whether it is used concomitantly with other therapeutic agents. Other factors affecting the dose administered to the subject include. e.g., the type or severity of the disease. For example, a subject having pancreatic cancer may require administration of a different dosage than a subject with brain cancer.

The effective amount of a compound (for example, a chemotherapeutic agent or an immunomodulator) described herein or pharmaceutically acceptable salts or prodrugs thereof can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day; about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner.

When administering immortalized MSC-RRV cells described herein, an effective amount of immortalized MSC-RRV cells described herein will vary and can be determined by one of skill in the art through experimentation and/or clinical trials. For example, for in vivo injection, an effective dose can be from about 5×10⁷ to 1.5×10⁸ MSC-RRV cells/dose See Fares et al. Lancet Oncol. 22 (8): 1103-1114 (2021)). Effective doses for any of the administration methods described herein can be extrapolated from dose-response curves derived from in vitro or animal model test systems.

As used herein, “administer” or “administration” refers to the act of introducing, injecting or otherwise physically delivering a substance as it exists outside the body (e.g. immortalized MSC-RRC cell(s)) into a subject, such as by mucosal, intradermal, intravenous, intratumoral, intraperitoneal, intramuscular, intrarectal, oral, subcutaneous, intranasal delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The immortalized MSC-RRV cells provided herein are administered via any of several routes of administration, including parenterally, intramucosally, intravenously, intratumorally, intraperitoneally, intraventricularly, intramuscularly, subcutaneously, intracranially, intracavity or transdermally. Administration can be achieved by, e.g., topical administration, local infusion, injection, or by means of an implant.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

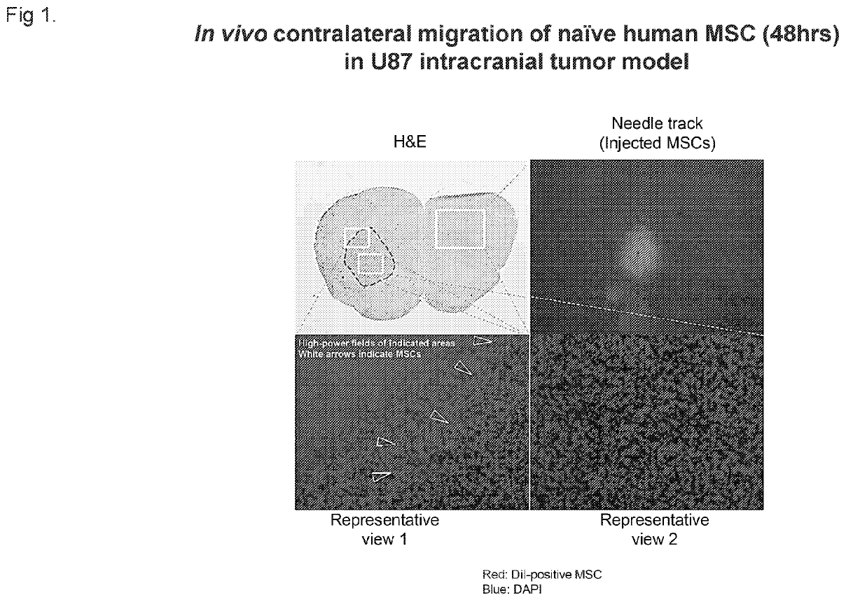

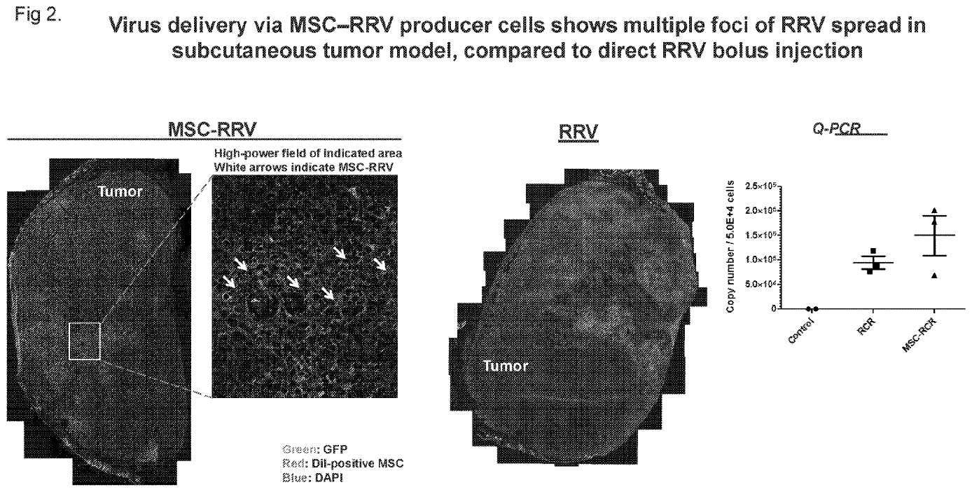

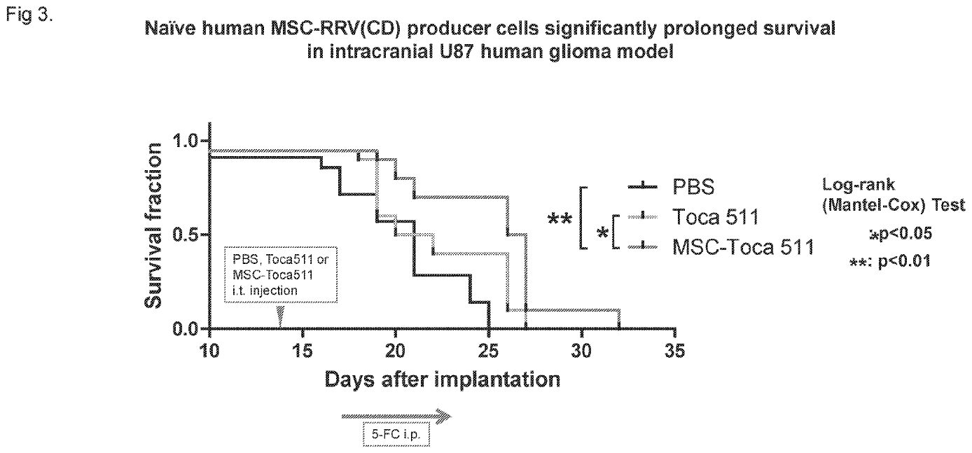

Retroviral replicating vectors (RRV) have been developed for efficient tumor-selective vector gene therapy for glioma. RRV-transduced tumor cells produce new virus progenies, which efficiently spread throughout the tumor mass from the injection site without any cytopathic effect. Cytotoxicity is triggered by conversion of non-toxic prodrug 5-FC to chemotherapeutic drug 5-FU mediated by vector-encoded transgene cytosine deaminase (CD). One limitation to the use of RRV for metastatic brain tumors, which are frequently multi-focal, is the need to inject each lesion individually. To overcome this potential limitation, studies were conducted where primary human mesenchymal stem cells (MSCs) were employed as tumor-homing cellular carriers that produce and deliver RRV to multiple tumor sites. It was demonstrated that MSCs migrated into the established intracranial human U87 glioma xenograft lesions from the contralateral injection side in athymic nude mice (FIG. 1). Furthermore, these results indicated that MSC engineered to serve as a RRV carrier and stable producer can achieve more efficient intratumoral gene delivery than direct RRV bolus injection, especially at lower multiplicity of infection (MOI) of 0.01 (FIG. 2). This increased tumor transduction efficiency by MSC RRV producer cells vs. RRV itself translated to an enhanced therapeutic efficacy in intracranial tumor model (FIG. 3).

However, extensive variability was observed among different primary human MSC isolates in their in vivo migration capabilities towards intracranial human glioma xenografts. Besides the unpredictability of in vivo tumor tropism, an additional obstacle to clinical applications of primary human MSCs is their limited expansion capability. Use of autologous adult patient-derived MSCs requires ex vivo expansion due to low yield of patient-derived MSCs. Primary human MSCs have a capacity to undergo about 30-40 population doublings (which translates to about 10-15 passages) due to the replicative senescence phenomenon and lack of hTERT expression. Practical constraints for using autologous adult patient-derived MSCs (e.g., donor-inter variability in cell availability, ex vivo expandability, in vivo migratory behavior, and tumor tropism) combined with the high cost of production of individual autologous MSC-based therapeutics and delayed timing of treatment (because of the time needed for autologous MSC expansion and virus infection) preclude their use in clinical applications. Therefore, an alternative strategy for generating therapeutic MSC-RRV cells is development of stable, immortalized “universally-histocompatible” human MSC-RRV clonal cells line as described herein.

### Generation of MSC-RRV Cells

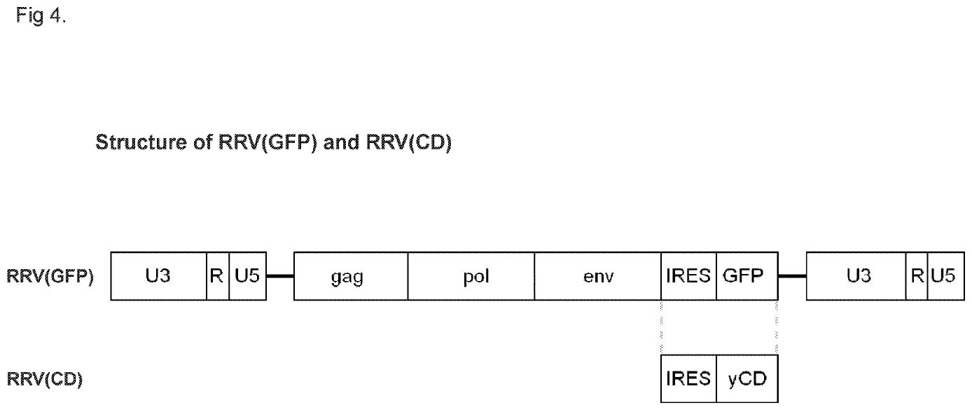

Generation of MSC-RRV cells (e.g., therapeutic MScs) as a carrier of RRV for glioma virotherapy requires production and validation of multiple immortalized MSC lines derived from different fetal, perinatal, or adult human MSC sources, and consequent selection for their self-renewal capabilities for substantial number of passages. RRV secretion capabilities, in vivo tumor tropism and minimal expression of HLA class I under inflammatory conditions must also be considered. To facilitate this effort, a protocol for generating a MSC-RRV line that produces RRV (GFP) (FIG. 4) and is immortalized by transduction with non-replicating lentiviral vectors (LV) expressing human L-MYC, human TERT, and shRNA targeting pan-Class I HLA (FIG. 5) was developed and validated.

LV expressing L-MYC from an EF1α promoter was constructed by recloning L-myc from plasmid pMXs-Hu-L-Myc (Addgene plasmid ID 26022), and LV expressing hTERT from an EF1α promoter was constructed by recloning hTERT from plasmid pBABE-neo-hTERT (Addgene plasmid ID 1774).

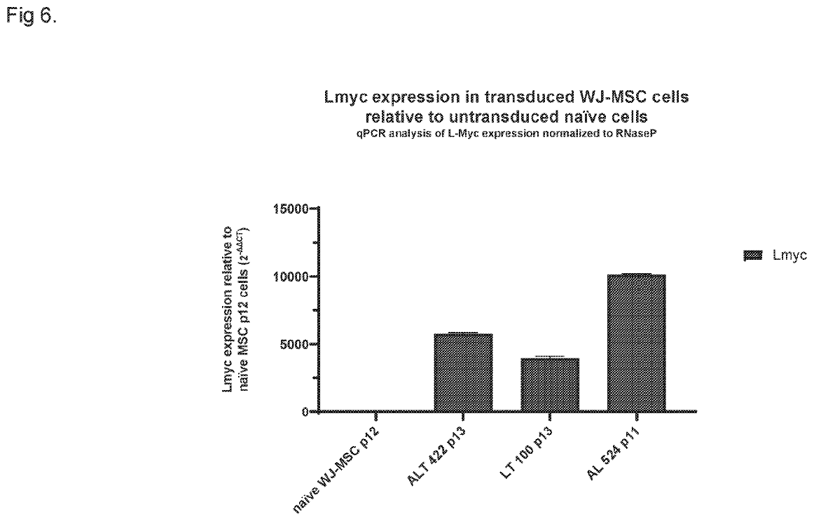

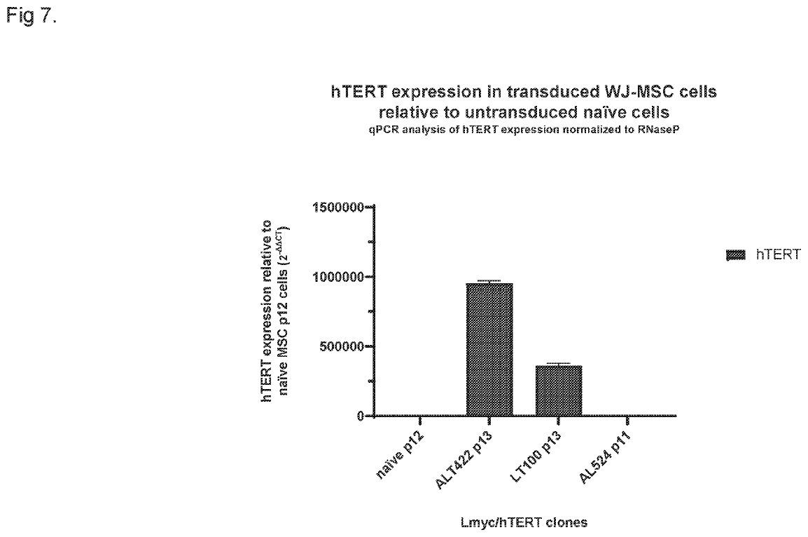

Human primary Wharton's Jelly-derived MSCs (WJ-MSC) were obtained from LifeLine Cell Technologies (#FC-0020) and maintained according to the manufacturer's protocol Briefly, WJ-MSCs were cultured under normoxic, 5% CO₂ conditions in a humidified incubator in a complete StemLife MSC medium (LifeLine Cell Technologies #LL-0034). Cells were seeded at 5,000 cells/cm² in tissue culture-treated 6-well plates and expanded for 4 days to 60-80% confluence. Medium was exchanged once in 4 days. At passage 4, WJ-MSC were transduced with RRV (GFP) virus at MOI 1 and cultured for 2 passages to allow for RRV spread throughout the MSC cells for 10 days. RRV (OFP)-transduced MSC cells were further double-transduced at passage 6 with LV encoding for L-myc gone and simultaneously with LV encoding for hTERT gene. Medium was exchanged within 12 hr, and 48 hr after transduction MSC cells were split 1: S and selection culture medium containing G418 (4 ng/ml) and puromycin (0.4 μg/ml) was added. Selection medium eliminated cells that failed to transduce with both L-myc and hTERT genes within 6 days. Cells continued to be cultured in selection medium for 28 days, followed by selection intermittently once every 4 passages. Emerging colonies were isolated and transferred into single wells in 6-well plates for expansion. L-MYC and hTERT expression in transduced clones was confirmed by qPCR (FIGS. 6 and 7). Cell morphology and growth rate was monitored over the subsequent passages by counting number of cells per well, and population doubling time (PDT) of every passage was calculated according to the formula provided at the website (https://www.doubling-time.com/compute.php)

Doubling Time=duration*log(2)/log (FinalConcentration)−log (InitalConcentration).

PDT of non-immortalized, naïve WJ-MSCs was monitored for determining passage number at which naïve MSCs undergo replicative senescence. Naïve MSCs stopped growing abruptly and synchronously at passage 16 with PDT 129, increased from PDT 45 at passage 15. Two, different potentially immortalized L-MYC+hTERT transduced clones with sustained growth and PDT lower than PDT of naïve cells were obtained: one is termed ALT422 (A stands for ‘AC3EMD’ RRV vector. L for L-MYC, and T for hTERT) and the other clone does not produce RRV and is termed LT100. Starting at p12, clone ALT422 consistently exhibited ≥98% viability and a superior sustained growth rate beyond passage 16 at PDT≤30 hours when medium was exchanged every 4 days, and ≤27 hours when medium was exchanged every 2 days. Compared to clone ALT422, clone LT100 maintained its PDT at ≤37 hours starting from passage 6.

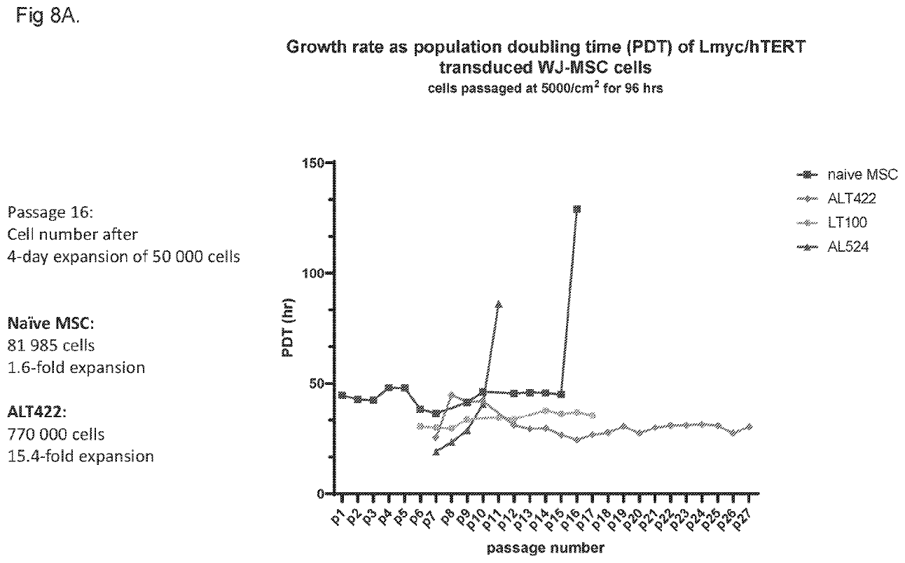

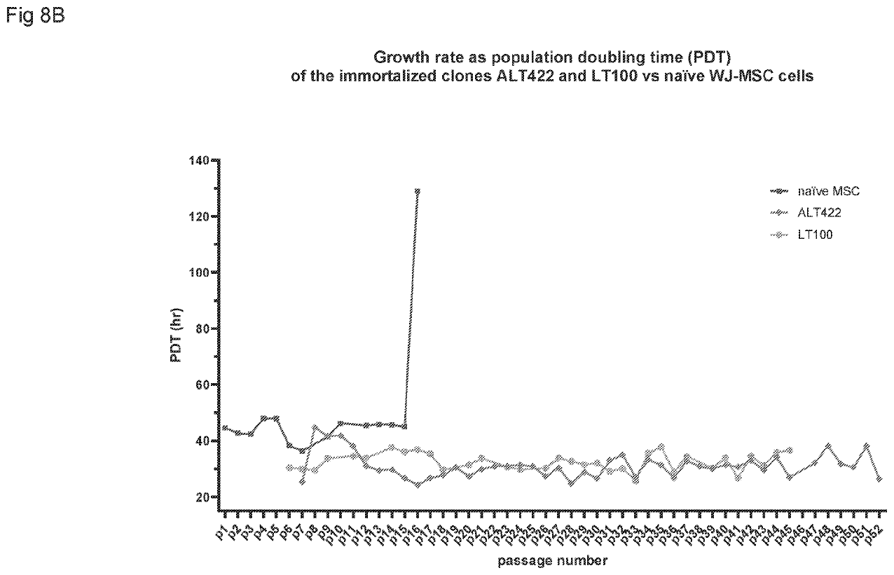

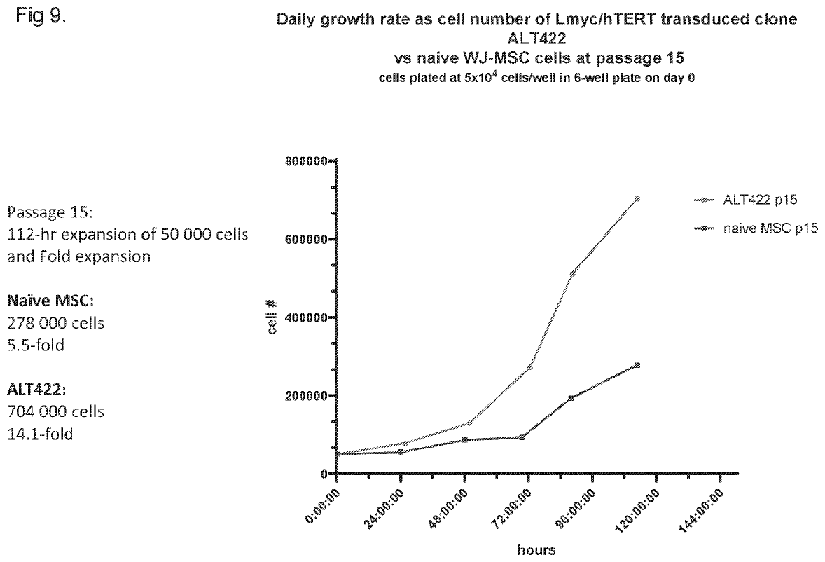

Clonal cell line ALT422 was considered immortalized because it stably maintains its robust self-renewal capabilities and can be propagated up to at least passage 27 (FIG. 8A), expands robustly 15-fold per passage (4 days), which exponentially increases the number of MSCs available for use in potential therapies. In comparison, naïve WJ-MSC expanded only 1.6-fold at passage 16 (FIG. 8A). A number of MSC-RRV clones transduced with L-MYC, but without hTERT, were also generated, and the best performing clone of these series, AL524 reached senescence at passage 11 with PDT of 86 hr. A lack of hTERT expression in clone AL524 was confirmed by qPCR (FIG. 7).

FIG. 8B shows that clonal cell line ALT422 stably maintains its robust self-renewal capabilities with PDT averaging 30.4 hours and can be propagated up to at least passage 52. Compared to clone ALT422, clone LT100 maintained its PDT at the average PDT 32.5 hours starting from passage 6, when it emerged, to at least passage 45.

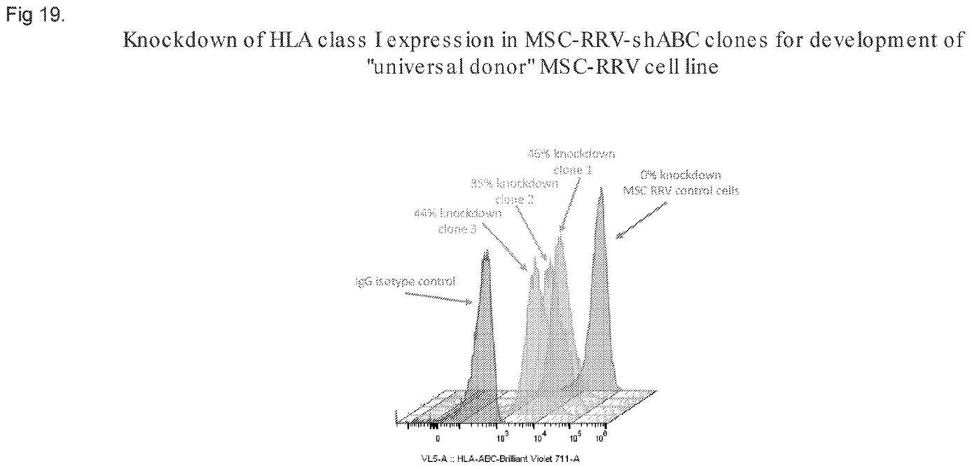

ALT422 line was further transduced with LV-shABC-ER vector to knockdown expression of HLA class I antigens. Evaluation of HLA-ABC antigens knockdown in WJ-MSC cells transduced with vector LV-shABC-ELTIN (FIG. 5) revealed a 35-46% knockdown of class I HLA (FIG. 19). As an alternative to the partial shRNA-mediated knockdown, a complete knockout of HLA class I antigens can be performed by CRISPR prime editing using integration-defective LV expressing PEmax-P2A-bMLHIdn recloned from Addgene plasmid ID 174828 pCMV-PEmax-P2A-hMLHIdn to LV under EF1α promoter.

Once the feasibility of immortalization by a combination of L-MYC and hTERT was determined, a single LV (termed LV-shABC-ELTIN) encoding two expression cassettes: U6 promoter-shRNA targeting HLA-A, B, C and EF1α promoter-L-MYC-P2A-hTERT-IRES-Neo^{R} (FIG. 5) was constructed. LV-shABC-ELTIN vector can be validated for immortalization and knockdown function in naïve WJ-MSC not transduced with RRV and in RRV (CD)-infected WJ-MSCs. The LV-shABC-ELTIN vector minimizes the multiple integration sites created by three separate LV vectors.

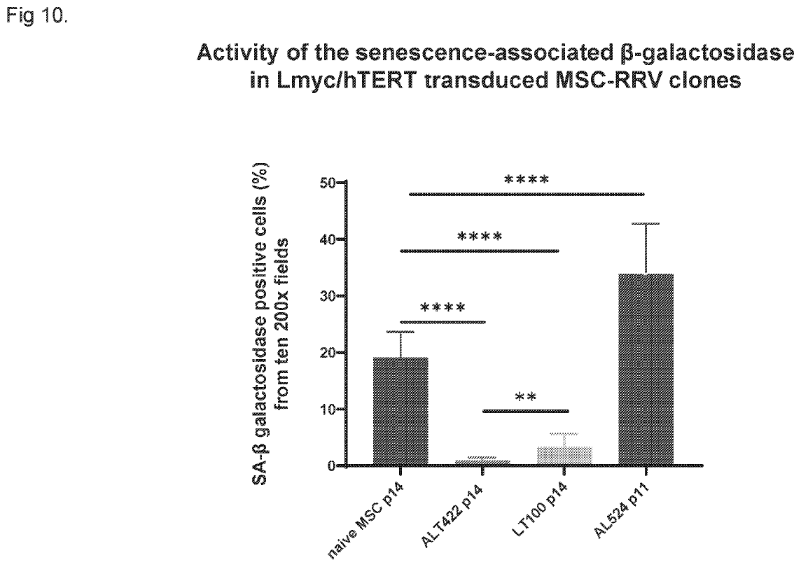

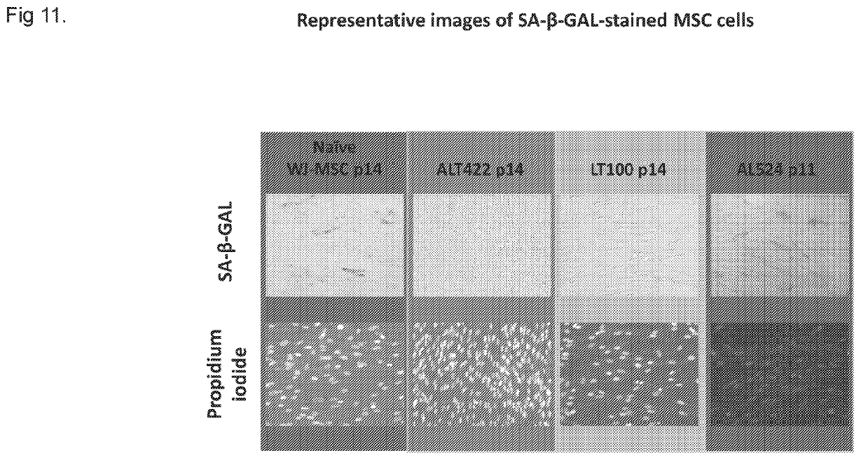

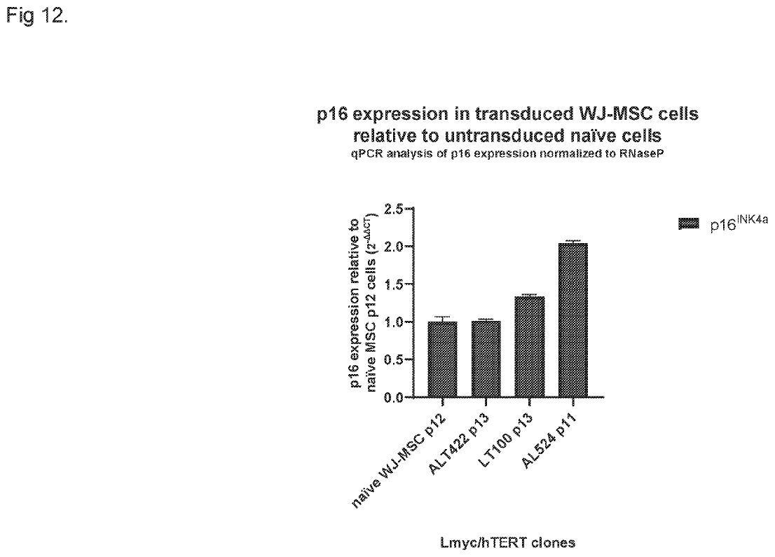

The immortalized state of clone ALT422 was confirmed by detection of β-galactosidase (β-GAL) activity, a histochemical assay that is called senescence-associated β-galactosidase (SA-β-GAL) because it labels senescent cells in vitro. Staining was performed according to the manufacturer's instructions (Cell Signaling Technology, Senescence β-Galactosidase Staining Kit #9860) and stained cells and total number of cells per field in 10 fields were counted. The percentage of the senescent cells per field were also calculated. Total number of cells was quantified by counting propidium iodide-stained nuclei in cells treated with RNaseH. SA-β-GAL assay revealed a presence of 0.9% β-GAL-positive cells in p14 ALT422 cell population, and a significant (t test, p value <0.0001) 20-fold higher percentage of β-GAL-positive cells in p14 naïve WJ-MSC cells (FIGS. 10 and 11). LT100 clone at p14 displayed 3.5-fold higher SA-β-GAL activity than clone ALT422. For comparison, hTERT-negative p11 AL524 clone (with PDT 86 hr at passage 11) was included and these cells showed SA-β-GAL positivity in 34% cells (vs. 19% in naïve WJ-MSC p14), indicating that AL524 clone reached senescence at p11 and that hTERT expression could be important for facilitating the establishment of the immortalized phenotype. Further, the expression of cell cycle inhibitor CDKN2A/p16 by qPCR was analyzed and, interestingly, SA-β-GAL positivity correlated with CDKN2A/p16 expression only in the LV-transduced cells, but not in naïve p12 WJ-MSC cells (FIG. 12), indicating that naïve MSC cells can suppress expression of CDKN2A/p16, at least at this passage number.

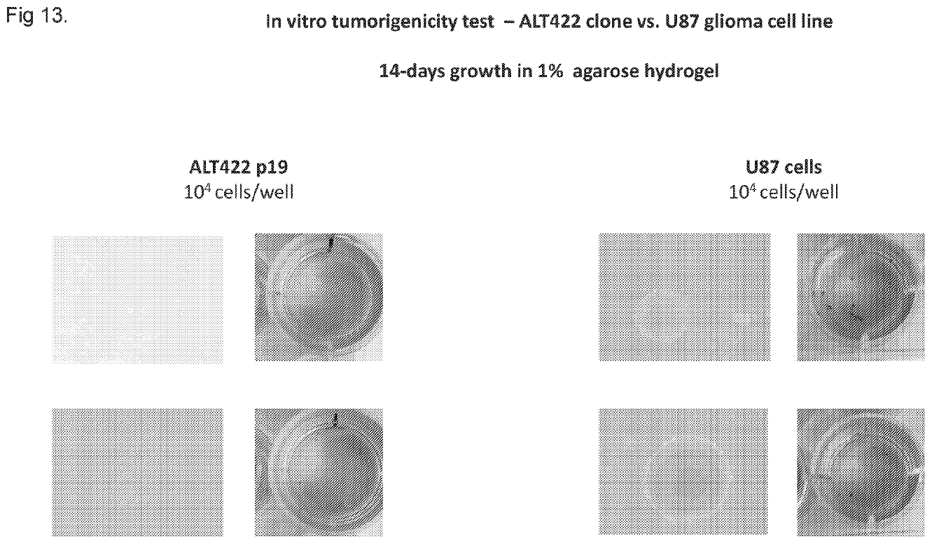

Soft agar colony formation assay is an in vitro tumorigenicity assay to assess cellular anchorage-independent growth of ALT422 line compared with U87 glioma cells. ALT422 p19, in contrast to the U87 cancer cell line, did not form colonies in soft 1% agar, but rather remained viable as single cells (FIG. 13).

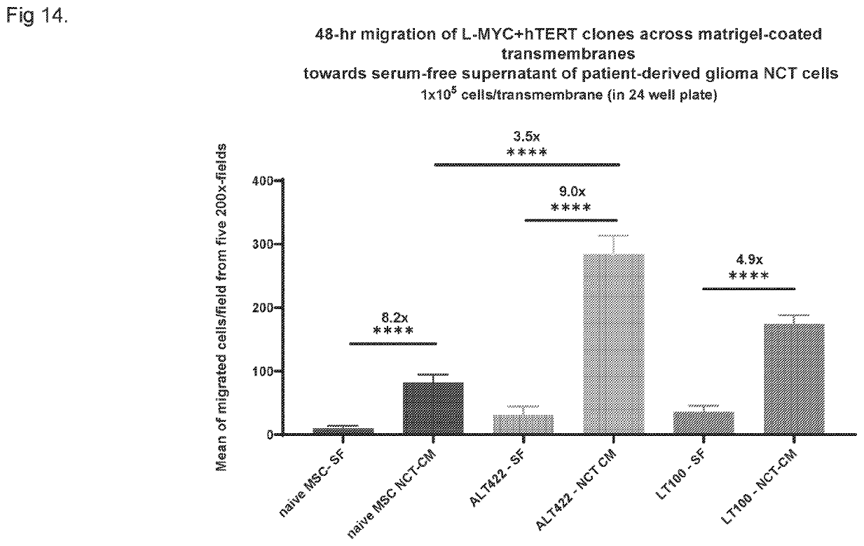

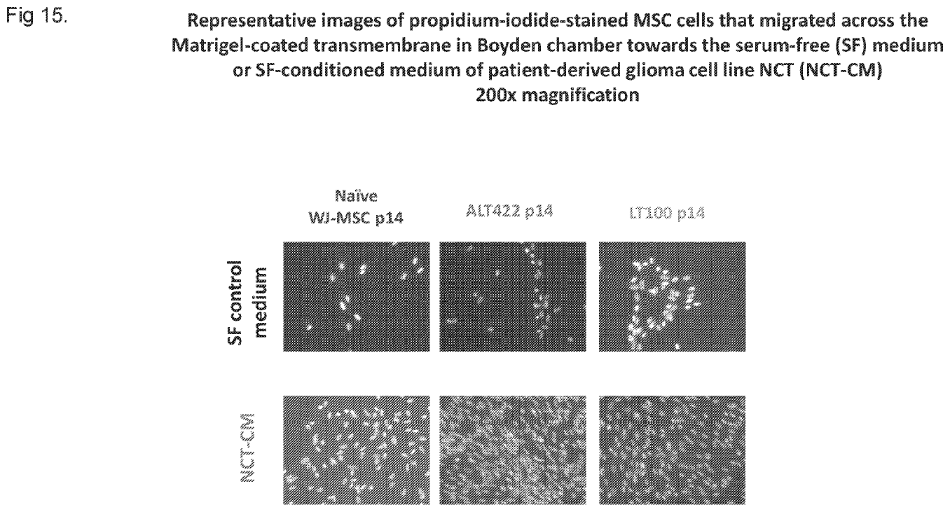

Cell motility and tropism of ALT422 cells to conditioned media from 2-days culture of patient-derived glioblastoma cell line NCT was assessed by Boyden chamber migration assay using 24-well cell-culture plates with 8 μm pore size polycarbonate transmembrane inserts (Corning Transwell #3422). Transmembranes were coated with a thin layer of 70 ul of growth-factor-reduced Matrigel at 100 μg/ml concentration for 4 hr. Coating of transmembranes with a thin layer of Matrigel allows MSC cells to attach to polycarbonate membrane material, which is important for ability of MSC cells to migrate. After coating with Matrigel. Matrigel was aspirated, and transmembranes were rinsed once with PBS and 1×10⁵ MSC cells were seeded onto the transwells in 300 μl complete StemLafe MSC medium. MSC cells were allowed to migrate across the membrane for 48 hr to the bottom compartment containing either 300 μl serum-free (SF) medium or 300 μl serum-free conditioned medium collected as a supernatant from 2-day culture of NCT cells (SF-CM). All tested MSC cells were at p14 and exhibited 5 to 9-fold increased migrating activity towards the glioblastoma conditioned medium when compared to migration towards the control SF medium. ALT422 cells bad the highest migratory activity towards SF-CM and 3.5-fold more cells migrated towards SF-CM compared to untransduced naïve cells. However, ALT422's migratory capacity towards SF control medium was also 3-fold higher when compared to naïve WI-MSC cells at the same passage 14 (FIG. 14, 15). Chemo-attractants present in the NCT conditioned medium stimulated migratory activity of naïve cells and ALT422 cells to the same degree. 8.2-fold and 9-fold, respectively.

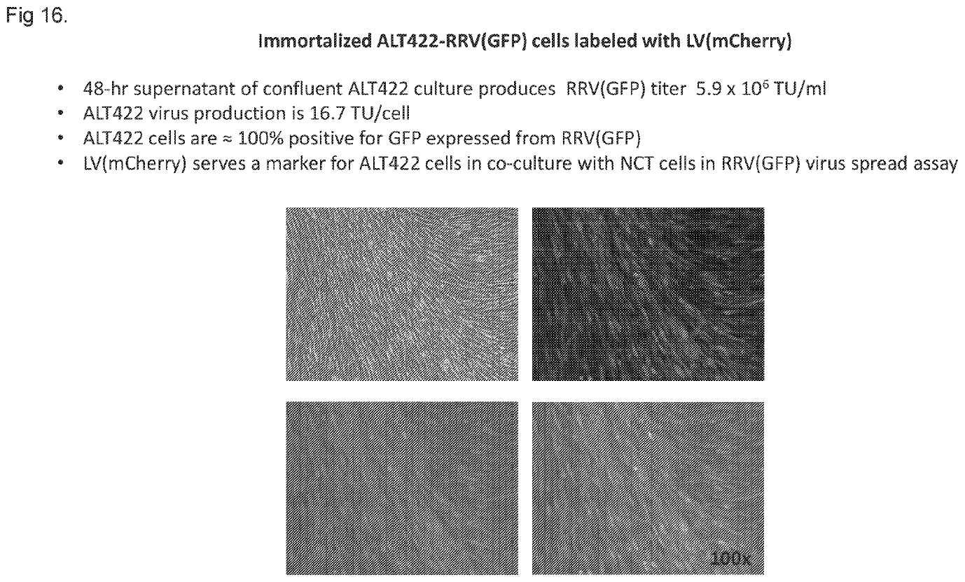

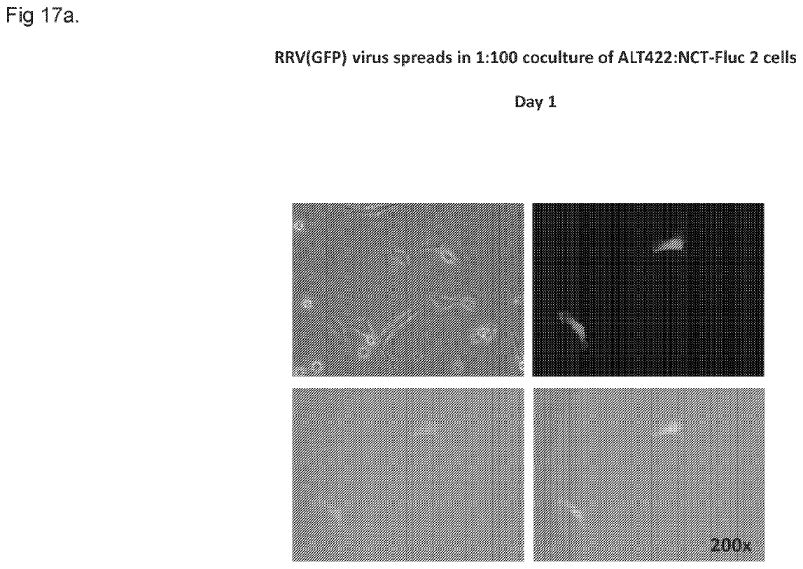

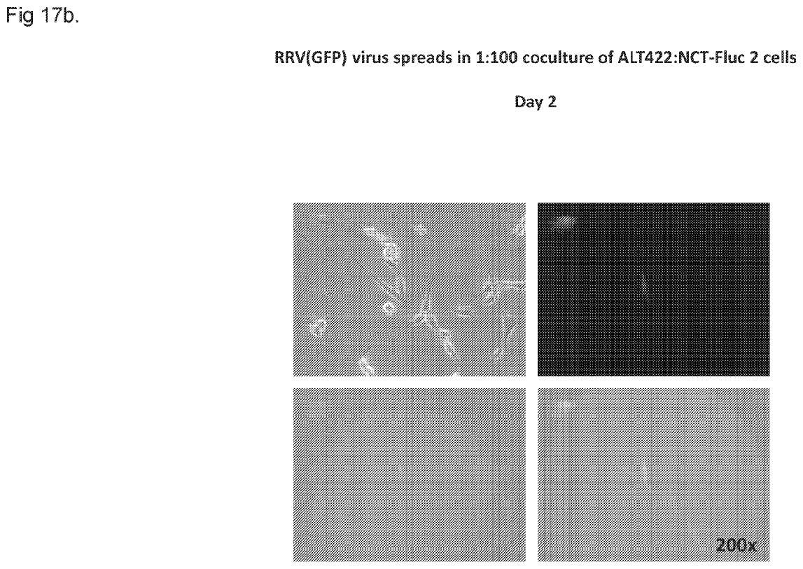

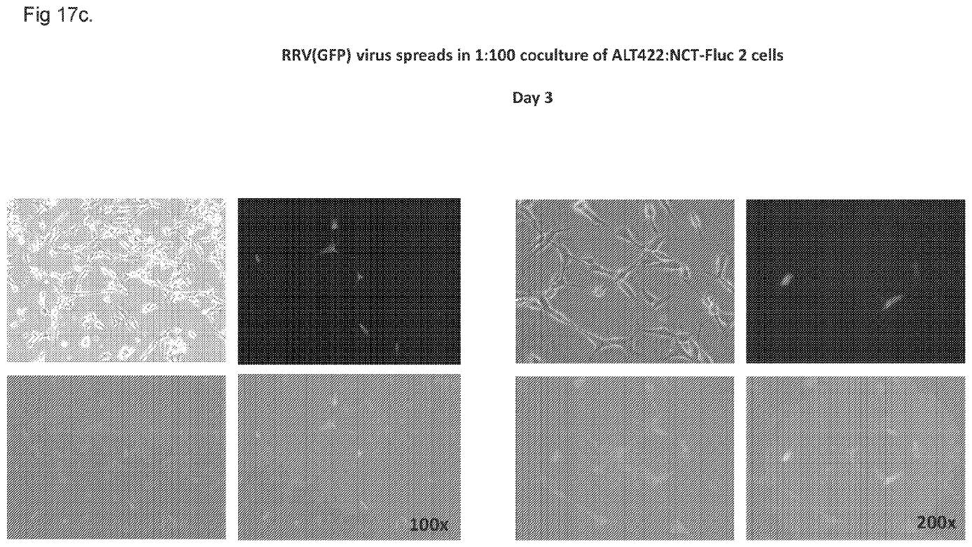

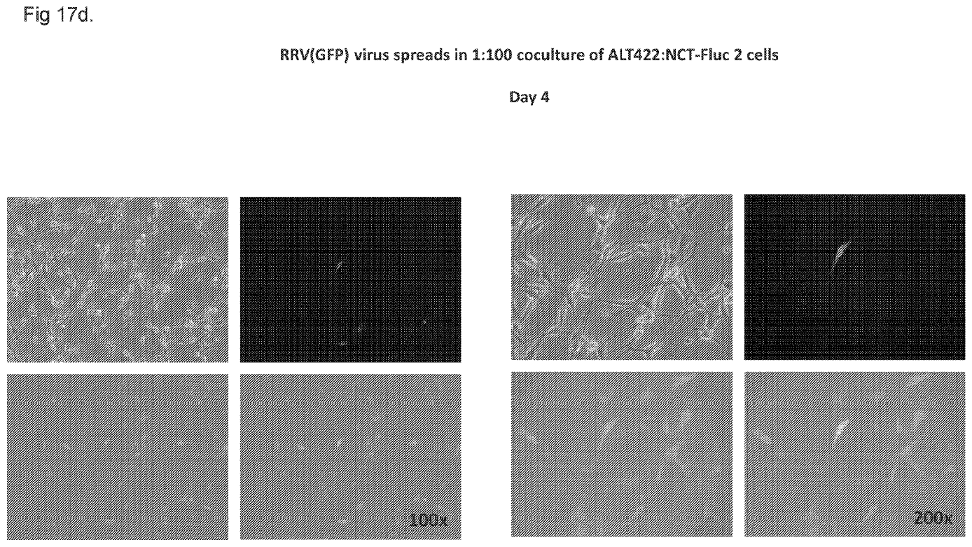

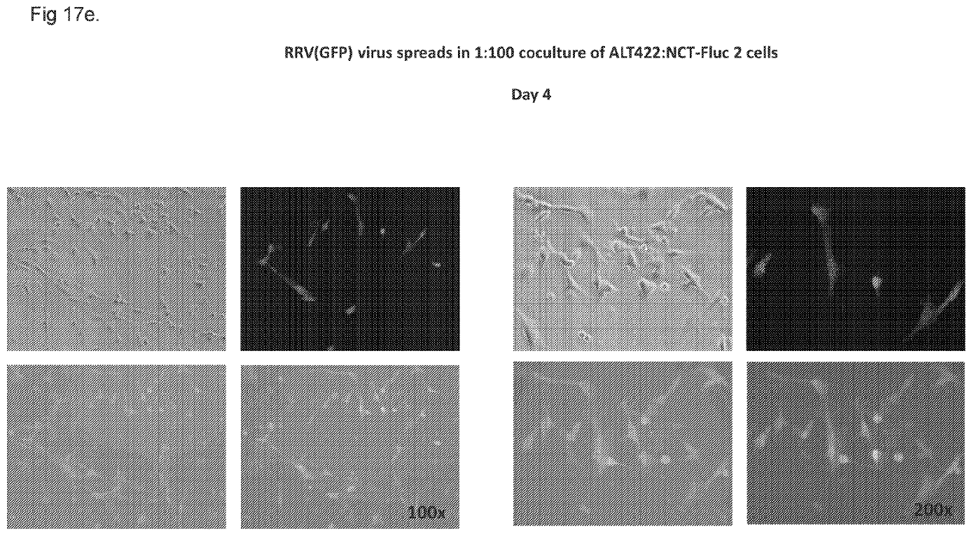

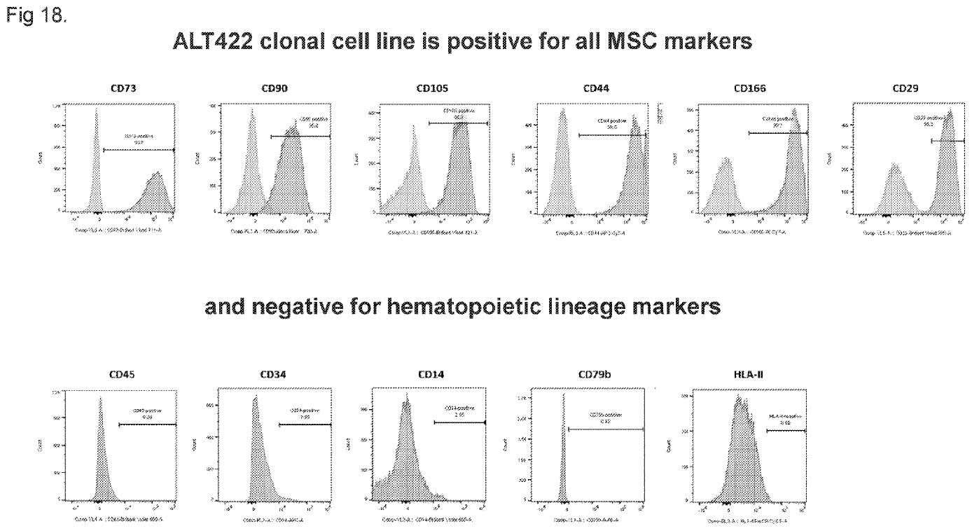

The ALT422 cell line was characterized for RRV (GFP) virus production levels and functionality of the released virus. A 48-hr supernatant harvested from a confluent ALT422 culture yielded high titer of 5.9×10⁶ TU/ml, which translates to functional viral particle production of 16.7 TU/cell (FIG. 16). RRV (GFP) virus was titrated on U87 cells. To analyze the efficiency of virus production and spread from ALT422 producer cells in NCT cell culture, co-culture experiments were performed, in which mCherry-labeled ALT422 cells (FIG. 16) transduced with non-replicating LV (mCherry) that allows for identification of ALT422 cells in co-culture with NCT cells were used. ALT422 (mCherry) cells were mixed with NCT cells at a ratio of 1:100. Virus spread in NCT cells was monitored daily for 4 days by detection of GFP-positive cells in the images overlapped with images of mCherry-positive cells. Fluorescent images show progressively more green-positive cells over a period of 4 days (FIG. 17a-e). GFP-positive NCT cells were already detected at day 2 (FIG. 17b). A widespread virus infection of NCT cells was detected at day 3 (FIG. 17c), with an increasing GFP intensity in the infected NCT cells at day 4 (FIG. 17d-e), thus showing that immortalized MSCs can be used as carriers for RRVs.

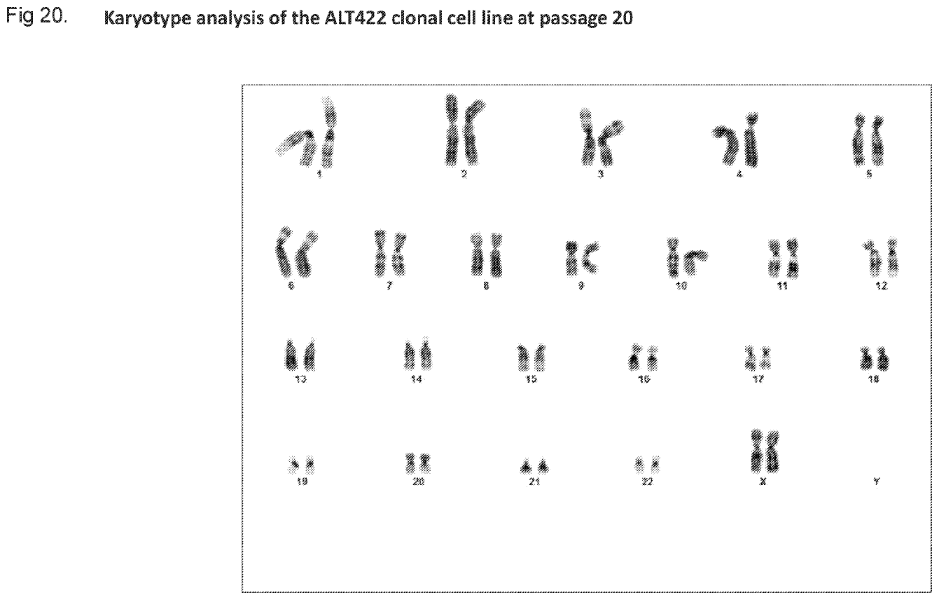

Cytogenetic analysis was performed on twenty G-banded metaphase spreads of the human cell line ALT422. Every spread displayed an apparently normal female karyotype 46, XX. Karyotype analysis of the ALT422 cell line at passage 20 is shown in FIG. 20.

As described herein, an MSC-RRV cell line that retains self-renewal capabilities beyond passage 21 (as opposed to non-immortalized MSC that uniformly reached replicative senescence at passage 16) was developed. In addition, this MSC-RRV cell line maintains high in vitro migratory activity towards serum-free conditioned medium of a patient-derived glioblastoma cell line. MSC-RRV growth rate, with a population doubling time ≤30 hours, correlates with inhibition of cell-cycle arrest markers, such as senescence-associated β-galactosidase (FIG. 10) and cell cycle inhibitor CDKN2A/p16. Further, the MSC-RRV line produces high RRV titers of 6×10⁶ TU/ml. Importantly, since MSC-RRV is tumor specific and can be eliminated in vivo by administration of prodrug 5-fluorocytosine (5-FC), MSC-RRV represents a therapeutic platform without systemic toxicity.

Sequences
L-Myc sequence (1095 nt)
SEQ ID NO: 1
Atggactacgactcgtaccagcactatttctacgactatgactgcggg
gaggatttctaccgctccacggcgcccagcgaggacatctggaagaaa
ttcgagctggtgccatcgccccccacgtcgccgccctggggcttgggt
cccggcgcaggggacccggcccccgggattggtcccccggagccgtgg
cccggagggtgcaccggagacgaagcggaatcccggggccactcgaaa
ggctggggcaggaactacgcctccatcatacgccgtgactgcatgtgg
agcggcttctcggcccgggaacggctggagagagctgtgagcgaccgg
ctcgctcctggcgcgccccgggggaacccgcccaaggcgtccgccgcc
ccggactgcactcccagcctcgaagccggcaacccggcgcccgccgcc
ccctgtccgctgggcgaacccaagacccaggcctgctccgggtccgag
agcccaagcgactcggagaatgaagaaattgatgttgtgacagtagag
aagaggcagtctctgggtattcggaagccggtcaccatcacggtgcga
gcagaccccctggatccctgcatgaagcatttccacatctccatccat
cagcaacagcacaactatgctgcccgttttcctccagaaagctgctcc
caagaagaggcttcagagaggggtccccaagaagaggttctggagaga
gatgctgcaggggaaaaggaagatgaggaggatgaagagattgtgagt
cccccacctgtagaaagtgaggctgcccagtcctgccaccccaaacct
gtcagttctgatactgaggatgtgaccaagaggaagaatcacaacttc
ctggagcgcaagaggcggaatgacctgcgttcgcgattcttggcgctg
agggaccaggtgcccaccctggccagctgctccaaggcccccaaagta
gtgatcctaagcaaggccttggaatacttgcaagccctggtgggggct
gagaagaggatggctacagagaaaagacagctccgatgccggcagcag
cagttgcagaaaagaattgcatacctcactggctactaa
hTERT sequence (3399 nt)
SEQ ID NO: 2
atgccgcgcgctccccgctgccgagccgtgcgctccctgctgcgcagc
cactaccgcgaggtgctgccgctggccacgttcgtgcggcgcctgggg
ccccagggctggcggctggtgcagcgcggggacccggcggctttccgc
gcgctggtggcccagtgcctggtgtgcgtgccctgggacgcacggccg
ccccccgccgccccctccttccgccaggtgtcctgcctgaaggagctg
gtggcccgagtgctgcagaggctgtgcgagcgcggcgcgaagaacgtg
ctggccttcggcttcgcgctgctggacggggcccgcgggggccccccc
gaggccttcaccaccagcgtgcgcagctacctgcccaacacggtgacc
gacgcactgcgggggagcggggcgtgggggctgctgctgcgccgcgtg
ggcgacgacgtgctggttcacctgctggcacgctgcgcgctctttgtg
ctggtggctcccagctgcgcctaccaggtgtgcgggccgccgctgtac
cagctcggcgctgccactcaggcccggcccccgccacacgctagtgga
ccccgaaggcgtctgggatgcgaacgggcctggaaccatagcgtcagg
gaggccggggtccccctgggcctgccagccccgggtgcgaggaggcgc
gggggcagtgccagccgaagtctgccgttgcccaagaggcccaggcgt
ggcgctgcccctgagccggagcggacgcccgttgggcaggggtcctgg
gcccacccgggcaggacgcgtggaccgagtgaccgtggtttctgtgtg
gtgtcacctgccagacccgccgaagaagccacctctttggagggtgcg
ctctctggcacgcgccactcccacccatccgtgggccgccagcaccac
gcgggccccccatccacatcgcggccaccacgtccctgggacacgcct
tgtcccccggtgtacgccgagaccaagcacttcctctactcctcaggc
gacaaggagcagctgcggccctccttcctactcagctctctgaggccc
agcctgactggcgctcggaggctcgtggagaccatctttctgggttcc
aggccctggatgccagggactccccgcaggttgccccgcctgccccag
cgctactggcaaatgcggcccctgtttctggagctgcttgggaaccac
gcgcagtgcccctacggggtgctcctcaagacgcactgcccgctgcga
gctgcggtcaccccagcagccggtgtctgtgcccgggagaagccccag
ggctctgtggcggcccccgaggaggaggacacagacccccgtcgcctg
gtgcagctgctccgccagcacagcagcccctggcaggtgtacggcttc
gtgcgggcctgcctgcgccggctggtgcccccaggcctctggggctcc
aggcacaacgaacgccgcttcctcaggaacaccaagaagttcatctcc
ctggggaagcatgccaagctctcgctgcaggagctgacgtggaagatg
agcgtgcgggactgcgcttggctgcgcaggagcccaggggttggctgt
gttccggccgcagagcaccgtctgcgtgaggagatcctggccaagttc
ctgcactggctgatgagtgtgtacgtcgtcgagctgctcaggtctttc
ttttatgtcacggagaccacgtttcaaaagaacaggctctttttctac
cggaagagtgtctggagcaagttgcaaagcattggaatcagacagcac
ttgaagagggtgcagctgcgggagctgtcggaagcagaggtcaggcag
catcgggaagccaggcccgccctgctgacgtccagactccgcttcatc
cccaagcctgacgggctgcggccgattgtgaacatggactacgtcgtg
ggagccagaacgttccgcagagaaaagagggccgagcgtctcacctcg
agggtgaaggcactgttcagcgtgctcaactacgagcgggcgcggcgc
cccggcctcctgggcgcctctgtgctgggcctggacgatatccacagg
gcctggcgcaccttcgtgctgcgtgtgcgggcccaggacccgccgcct
gagctgtactttgtcaaggtggatgtgacgggcgcgtacgacaccatc
ccccaggacaggctcacggaggtcatcgccagcatcatcaaaccccag
aacacgtactgcgtgcgtcggtatgccgtggtccagaaggccgcccat
gggcacgtccgcaaggccttcaagagccacgtctctaccttgacagac
ctccagccgtacatgcgacagttcgtggctcacctgcaggagaccagc
ccgctgagggatgccgtcgtcatcgagcagagctcctccctgaatgag
gccagcagtggcctcttcgacgtcttcctacgcttcatgtgccaccac
gccgtgcgcatcaggggcaagtcctacgtccagtgccaggggatcccg
cagggctccatcctctccacgctgctctgcagcctgtgctacggcgac
atggagaacaagctgtttgcggggattcggcgggacgggctgctcctg
cgtttggtggatgatttcttgttggtgacacctcacctcacccacgcg
aaaaccttcctcaggaccctggtccgaggtgtccctgagtatggctgc
gtggtgaacttgcggaagacagtggtgaacttccctgtagaagacgag
gccctgggtggcacggcttttgttcagatgccggcccacggcctattc
ccctggtgcggcctgctgctggatacccggaccctggaggtgcagagc
gactactccagctatgcccggacctccatcagagccagtctcaccttc
aaccgcggcttcaaggctgggaggaacatgcgtcgcaaactctttggg
gtcttgcggctgaagtgtcacagcctgtttctggatttgcaggtgaac
agcctccagacggtgtgcaccaacatctacaagatcctcctgctgcag
gcgtacaggtttcacgcatgtgtgctgcagctcccatttcatcagcaa
gtttggaagaaccccacatttttcctgcgcgtcatctctgacacggcc
tccctctgctactccatcctgaaagccaagaacgcagggatgtcgctg
ggggccaagggcgccgccggccctctgccctccgaggccgtgcagtgg
ctgtgccaccaagcattcctgctcaagctgactcgacaccgtgtcacc
tacgtgccactcctggggtcactcaggacagcccagacgcagctgagt
cggaagctcccggggacgacgctgactgccctggaggccgcagccaac
ccggcactgccctcagacttcaagaccatcctggactga
shRNA targeting pan-class I (underlined sequences
SEQ ID NO: 3
are inverted repeats that form a loop structure
gctactacaaccagagcgagtgtgctgtcctcgctctggttgtagtagc

What is claimed is:

1. An immortalized mesenchymal stem cell comprising a replicating retrovirus (RRV), wherein the RRV comprises:
a, a nucleic acid encoding a retroviral gag protein, a retroviral pol protein and a retroviral env protein; and
b. an expression cassette comprising a first regulatory sequence operably linked to a first heterologous nucleic acid sequence encoding a heterologous polypeptide.

2. The immortalized mesenchymal stem cell of claim 1, wherein the replicating retrovirus is selected from the group consisting of, murine leukemia virus (MLV), Moloney murine leukemia virus (MoMLV), Gibbon ape leukemia virus (GALV), Feline Leukemia virus (FeLV), RD114, and xenotropic XMLV.

3. The immortalized mesenchymal stem cell of claim 1 or 2, wherein the mesenchymal stem cell comprises a non-replicating lentivirus comprising an expression cassette comprising a second regulatory sequence operably linked to a second heterologous nucleic acid sequence encoding L-MYC and/or TERT.

4. The immortalized mesenchymal stem cell of any one of claims 1-3, wherein the mesenchymal stem cell has reduced Class I HLA expression as compared to a naïve mesenchymal stem cell.

5. The immortalized mesenchymal stem cell of any one of claims 1-4, wherein the first heterologous nucleic acid sequence encodes a prodrug activator.

6. The immortalized mesenchymal stem cell of claim 5, wherein the prodrug activator is selected from the group consisting of a cytosine deaminase, a herpes simplex virus thymidine kinase, nitroreductase and cytochrome P450.

7. The immortalized mesenchymal stem cell of any one of claims 3-6, wherein the second heterologous nucleic acid further comprises a nucleic acid sequence encoding a selectable marker.

8. The immortalized mesenchymal stem cell of any one of claims 1-7, wherein the first or second regulatory nucleic acid sequence is selected from the group consisting of a promoter, a self-cleaving peptide, an enhancer, and an internal ribosome entry site.

9. The immortalized mesenchymal stem cell of claim 8, wherein the promoter is an constitutive or an inducible promoter.

10. A method for making an immortalized mesenchymal stem cell comprising a replicating retrovirus (RRV), comprising:
a. transducing the immortalized mesenchymal stem cell with a replicating retrovirus (RRV), wherein the RRV comprises:
i. nucleic acid encoding a retroviral gag protein a retroviral pol protein and a retroviral env protein; and
ii. an expression cassette comprising a first regulatory sequence operably linked to a first heterologous nucleic acid sequence encoding a heterologous polypeptide; and
b. immortalizing the mesenchymal stem cell.

11. The method of claim 10, wherein the retrovirus is selected from the group consisting of, murine leukemia virus (MLV), Moloney murine leukemia virus (MoMLV), Gibbon ape leukemia virus (GALV), Feline Leukemia virus (FeLV), RD114, and xenotropic XMLV.

12. The method of claim 10 or 11, wherein immortalization comprises transducing the mesenchymal stem cell with a non-replication lentivirus comprising a second heterologous nucleic acid sequence encoding L-MYC and/or TERT.

13. The method of claim 12, wherein the method further comprises transducing the mesenchymal stem cell with a nucleic acid sequence that reduces Class 1 HLA expression in the mesenchymal cell.

14. The method of claim 13, wherein the nucleic acid sequence that reduces Class I HLA expression is a short hairpin RNA.

15. The method of any one of claims 10-14, wherein the first heterologous nucleic acid sequence in the RRV encodes a prodrug activator.

16. The method of claim 15, wherein the prodrug activator is selected from the group consisting of a cytosine deaminase, a herpes simplex virus thymidine kinase, nitroreductase and cytochrome P450.

17. The method of any one of claims 10-16, wherein the first or second regulatory nucleic acid sequence is selected from the group consisting of a promoter, an enhancer, a self-cleaving peptide, and an internal ribosome entry site.

18. The method of claim 17, wherein the promoter is an constitutive or an inducible promoter.

19. An immortalized mesenchymal stem cell or population of immortalized mesenchymal stem cells produced by the method of any one of claims 10-18.

20. A pharmaceutical composition comprising the immortalized mesenchymal stem cell or population of immortalized mesenchymal stem cells of any one of claim 1-9 or 19.

21. A method of treating a disease in a subject in need thereof comprising administering the immortalized mesenchymal stem cell or population of immortalized mesenchymal stem cells of any one of claim 1-9 or 19, or the pharmaceutical composition of claim 20, to the subject.

22. The method of claim 21, wherein the disease is a cell proliferative disorder.

23. The method of claim 22, wherein the cell proliferative disorder is selected from the group consisting of lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, and brain cancer including glioblastoma, anaplastic astrocytoma, oligodendroglioma, medulloblastoma.

24. The method of claim 23, wherein the cancer is glioblastoma.

25. The method of any one of claims 21-24, wherein the subject is a mammal.

26. The method of claim 25, wherein the subject is a human.

27. The method of any of claims 21-26, wherein administration is systemic, topical or local administration.

28. The method of any one of claims 21-27, wherein the method further comprises administering a second therapeutic agent to the subject.

29. The method of claim 28, wherein the second therapeutic agent is a nontoxic prodrug that is converted to a toxic drug by the prodrug activator.

30. The method of claim 29, wherein the second therapeutic agent is a chemotherapeutic agent.


SEQUENCE LISTING (3 sequences)

SEQ ID NO: 1 - DNA, 1095 bp, Synthetic construct
  source 1..1095: mol_type = other DNA
atggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaa

SEQ ID NO: 2 - DNA, 3399 bp, Synthetic construct
  source 1..3399: mol_type = other DNA
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 3 - DNA, 49 bp, Synthetic construct
  source 1..49: mol_type = other DNA
gctactacaaccagagcgagtgtgctgtcctcgctctggttgtagtagc